(19) United States
(12) Patent Application Publication
(10) Pub. No.: US 2022/0259596 A1
(43) Pub. Date: Aug. 18, 2022

(54) Inhibitors of microRNA 451a for Treatment of Endometriosis

(71) Applicant: YALE UNIVERSITY, New Haven, CT (US)

(72) Inventor: Hugh Taylor, Easton, CT (US)

(21) Appl. No.: 17/627,927

(22) PCT Filed: Jul. 17, 2020
(86) PCT No.: PCT/US20/42464

§ 371 Date: Jan. 18, 2022

Related U.S. Application Data
(60) Provisional application No. 62/876,430, filed on Jul. 19, 2019.

(51) Int. Cl. C12N 15/113

(52) U.S. Cl. CPC C12N 15/113; C12N 2310/122; C12N 2310/141; C12N 2310/113

(57) ABSTRACT

The invention includes compositions and methods for the treating or preventing endometriosis in a subject in need thereof. In one aspect, the invention relates to compositions and methods for inhibiting microRNA451a.

### CROSS REFERENCE TO RELATED APPLICATIONS

This application claims priority to U.S. Provisional Application No. 62/876,430, filed Jul. 19, 2019 which is hereby incorporated by reference herein in its entirety.

### STATEMENT OF GOVERNMENT SUPPORT

This invention was made with government support under HD076422 awarded by National Institutes of Health. The government has certain rights in the invention.

### BACKGROUND

Endometriosis is an estrogen-dependent pro-inflammatory disease, and 6-10% of reproductive age women suffer from infertility and pelvic pain resulting from endometriosis. Due to the chronic morbidity associated with this gynecological disorder, past studies have attempted to identify distinguishing molecular features of the endometriotic lesions with the aim of developing more effective prognostic, diagnostic and/or treatment strategies for the clinical management (Falcone et al., Obstet Gynecol. 2018. 131(3): 557-571; Nematian et al., J Clin Endocrinol Metab. 2018. 103(1): 64-74). Despite such efforts, however, many current clinical treatments are inadequate for symptom relief and have unacceptable side effects (Casper et al., Fertil Steril. 2017. 107(3): 521-522). All current treatments target sex steroids and none are disease specific. A precision medicine approach to endometriosis may allow treatment for endometriosis without the adverse effects of hormone modification such as impaired fertility, vasomotor symptoms or bone loss.

MicroRNAs (miRNAs) are endogenous, short, noncoding, functional RNAs that regulate gene expression either by translational repression or degradation of messenger RNA (mRNA) transcripts. Numerous non-coding RNAs including miRNAs are expressed in endometrium and endometriosis (Pan et al., Mol Hum Reprod. 2007. 13(11): 797-806; Ghazal et al., EMBO Mol Med. 2015. 7(8): 996-1003). Differential expression of multiple miRNAs have been identified between eutopic endometrium of women with and without endometriosis, in the circulation of women with and without endometriosis and in murine experimental endometriosis, and between eutopic and ectopic endometrial tissues from women with endometriosis.

However, there remains a need in the art for effective therapeutics for the treatment of endometriosis that do not lead to the hormonal side effects associated with current therapies. The present disclosure satisfies this unmet need.

### SUMMARY

In one embodiment, the invention relates to a method of treating or preventing endometriosis in a subject in need thereof, comprising administering to the subject an effective amount of an inhibitor of microRNA 451a (miR451a). In one embodiment, the inhibitor is at least one selected form the group consisting of a polypeptide, a nucleic acid, an aptamer, an anti-miR, antagomiR, a miR sponge, a silencing RNA (siRNA), a short hairpin RNA (shRNA), a morpholino, a piwi-interacting RNA (piRNA), a repeat associated small interfering RNA (rasiRNAs), and a small molecule.

In one embodiment, the inhibitor is an antisense nucleic acid molecule to miR451a. In one embodiment, the inhibitor comprises the sequence

(SEQ ID NO: 1)
AAACCGUUACCAUUACUGAGUU.

In one embodiment, the invention relates to a composition for treating endometriosis comprising an inhibitor of microRNA 451a (miR451a). In one embodiment, the inhibitor is at least one selected form the group consisting of a polypeptide, a nucleic acid, an aptamer, an anti-miR, antagomiR, a miR sponge, a silencing RNA (siRNA), a short hairpin RNA (shRNA), a morpholino, a piwi-interacting RNA (piRNA), a repeat associated small interfering RNA (rasiRNAs), and a small molecule.

In one embodiment, the inhibitor is an antisense nucleic acid molecule to miR451a. In one embodiment, the inhibitor comprises the sequence

(SEQ ID NO: 1)
AAACCGUUACCAUUACUGAGUU.

### BRIEF DESCRIPTION OF THE DRAWINGS

The following detailed description of preferred embodiments of the disclosure will be better understood when read in conjunction with the appended drawings. For the purpose of illustrating the disclosure, there are shown in the drawings embodiments which are presently preferred. It should be understood, however, that the disclosure is not limited to the precise arrangements and instrumentalities of the embodiments shown in the drawings.

FIG. 1 is a schematic depicting the systemic administration of miRNA in a mouse endometriosis model. After induction of endometriosis, mice received different treatment regimens of miR-451 inhibitors or miRNA negative control by intravenous.

FIG. 2A and FIG. 2B, depict an exemplary analysis of the macroscopic size of the endometriosis lesions in the murine model. FIG. 2A depicts images of the size of exemplary endometriosis lesions in the murine model. FIG. 2B depicts a comparison of total lesion size between the two groups, including fluid-filled cystic areas. Volume=(smallest diameter²×largest diameter)*π/6(mm³). Data are presented as mean±SEM; *P=0.004.

FIG. 3A and FIG. 3B depict the effect of miR-451 inhibitor treatment on mRNA expression of selected genes involved in the pathophysiology of endometriosis as determined by qRT-PCR. FIG. 3A depicts exemplary results demonstrating that miR-451a inhibitor treatment resulted in significant increases in the expression levels of YWHAZ, CAP39, MAPK1, β-catenin and IL-6, relative to the control group. FIG. 3B depicts exemplary results demonstrating that expression of MIF, cyclin-D1, TNF-α, and TLR-4 were unchanged. Data are presented as mean±SEM; *P<0.05

### DETAILED DESCRIPTION

The present invention relates to compositions and methods for treating and preventing endometriosis. For example, in certain aspects, the present inventions provide compositions for reducing lesion growth.

In one embodiment, the invention relates to modulation of the activity of miR451a for the treatment or prevention of endometriosis. For example, in one embodiment, the invention relates to compositions and methods for inhibiting the expression or activity of miR451a. For example, it is described herein that inhibiting the expression or activity of miR451a results in reduced endometriosis lesion size. It is further demonstrated that miR451a inhibitors treat endometriosis and simultaneously affects multiple pathways driving endometriosis without systemic hormonal side effects.

In one embodiment, invention relates to compositions and methods for inhibiting the expression or activity of miR451a. For example, in one embodiment, the present invention provides compositions comprising an inhibitor of miR451a. In one embodiment, the present invention provides methods for treating and preventing endometriosis comprising administering an inhibitor of miR451a.

### Definitions

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

The term “analog” as used herein generally refers to compounds that are generally structurally similar to the compound of which they are an analog, or “parent” compound. Generally, analogs will retain some characteristics of the parent compound, e.g., a biological or pharmacological activity. An analog may lack other, less desirable characteristics, e.g., antigenicity, proteolytic instability, toxicity, and the like. An analog includes compounds in which a particular biological activity of the parent is reduced, while one or more distinct biological activities of the parent are unaffected in the “analog.” As applied to polypeptides, the term “analog” may have varying ranges of amino acid sequence identity to the parent compound, for example at least about 70%, at least about 80%-85%, at least about 86%-89%, at least about 90%, at least about 92%, at least about 94%, at least about 96%, at least about 98% or at least about 99% of the amino acids in a given amino acid sequence of the parent or a selected portion or domain of the parent. As applied to polypeptides, the term “analog” generally refers to polypeptides which are comprised of a segment of about at least 3 amino acids that has substantial identity to at least a portion of a binding domain fusion protein. Analogs typically are at least 5 amino acids long, at least 20 amino acids long or longer, at least 50 amino acids long or longer, at least 100 amino acids long or longer, at least 150 amino acids long or longer, at least 200 amino acids long or longer, and more typically at least 250 amino acids long or longer. Some analogs may lack substantial biological activity but may still be employed for various uses, such as for raising antibodies to predetermined epitopes, as an immunological reagent to detect and/or purify reactive antibodies by affinity chromatography, or as a competitive or noncompetitive agonist, antagonist, or partial agonist of a binding domain fusion protein function. As applied to polynucleotides, the term “analog” may have varying ranges of nucleic acid sequence identity to the parent compound, for example at least about 70%, at least about 80%-85%, at least about 86%-89%, at least about 90%, at least about 92%, at least about 94%, at least about 96%, at least about 98% or at least about 99% of the nucleic acids in a given nucleic acid sequence of the parent or a selected portion or domain of the parent. As applied to polynucleotides, the term “analog” generally refers to polynucleotides which are comprised of a segment of about at least 9 nucleic acids that has substantial identity to at least a portion of the parent. Analogs typically are at least 15 nucleic acids long, at least 60 nucleic acids long or longer, at least 150 nucleic acids long or longer, at least 300 nucleic acids long or longer, at least 450 nucleic acids long or longer, at least 600 nucleic acids long or longer, and more typically at least 750 nucleic acids long or longer. Some analogs may lack substantial biological activity but may still be employed for various uses, such as for encoding epitopes for raising antibodies to predetermined epitopes, as a reagent to detect and/or purify sequences by hybridization assays, or as a competitive or noncompetitive agonist, antagonist, or partial agonist of a target or modulator of a target.

“Antisense,” as used herein, refers to a nucleic acid sequence which is complementary to a target sequence, such as, by way of example, complementary to a target miRNA sequence, including, but not limited to, a mature target miRNA sequence, or a sub-sequence thereof. Typically, an antisense sequence is fully complementary to the target sequence across the full length of the antisense nucleic acid sequence.

The term “body fluid” or “bodily fluid” as used herein refers to any fluid from the body of an animal. Examples of body fluids include, but are not limited to, plasma, serum, blood, lymphatic fluid, cerebrospinal fluid, synovial fluid, urine, saliva, mucous, phlegm and sputum. A body fluid sample may be collected by any suitable method. The body fluid sample may be used immediately or may be stored for later use. Any suitable storage method known in the art may be used to store the body fluid sample: for example, the sample may be frozen at about −20° C. to about −70° C. Suitable body fluids are acellular fluids. “Acellular” fluids include body fluid samples in which cells are absent or are present in such low amounts that the miRNA level determined reflects its level in the liquid portion of the sample, rather than in the cellular portion. Such acellular body fluids are generally produced by processing a cell-containing body fluid by, for example, centrifugation or filtration, to remove the cells. Typically, an acellular body fluid contains no intact cells however, some may contain cell fragments or cellular debris. Examples of acellular fluids include plasma or serum, or body fluids from which cells have been removed.

As used herein, the term “cell-free” refers to the condition of the nucleic acid as it appeared in the body directly before the sample is obtained from the body. For example, nucleic acids may be present in a body fluid such as blood or saliva in a cell-free state in that they are not associated with a cell. However, the cell-free nucleic acids may have originally been associated with a cell, such as an endometrial cell prior to entering the bloodstream or other body fluid. In contrast, nucleic acids that are solely associated with cells in the body are generally not considered to be “cell-free.” For example, nucleic acids extracted from a cellular sample are generally not considered “cell-free” as the term is used herein.

The term “clinical factors” as used herein, refers to any data that a medical practitioner may consider in determining a diagnosis or prognosis of disease. Such factors include, but are not limited to, the patient's medical history, a physical examination of the patient, complete blood count, analysis of the activity of enzymes, examination of cells, cytogenetics, and immunophenotyping of blood cells.

“Complementary” as used herein refers to the broad concept of subunit sequence complementarity between two nucleic acids. When a nucleotide position in both of the molecules is occupied by nucleotides normally capable of base pairing with each other, then the nucleic acids are considered to be complementary to each other at this position. Thus, two nucleic acids are substantially complementary to each other when at least about 50%, preferably at least about 60% and more preferably at least about 80% of corresponding positions in each of the molecules are occupied by nucleotides which normally base pair with each other (e.g., A:T and G:C nucleotide pairs).

As used herein, “conjugated” refers to covalent attachment of one molecule to a second molecule.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of a mRNA molecule also consists of the nucleotide residues of the mRNA molecule which are matched with an anti-codon region of a transfer RNA molecule during translation of the mRNA molecule or which encode a stop codon. The coding region may thus include nucleotide residues comprising codons for amino acid residues which are not present in the mature protein encoded by the mRNA molecule (e.g., amino acid residues in a protein export signal sequence).

The term “comparator” describes a material comprising none, or a normal, low, or high level of one of more of the marker (or biomarker) expression products of one or more the markers (or biomarkers) of the invention, such that the comparator may serve as a control or reference standard against which a sample can be compared.

As used herein, the term “derivative” includes a chemical modification of a polypeptide, polynucleotide, or other molecule. In the context of this invention, a “derivative polypeptide,” for example, one modified by glycosylation, pegylation, or any similar process, retains binding activity. For example, the term “derivative” of binding domain includes binding domain fusion proteins, variants, or fragments that have been chemically modified, as, for example, by addition of one or more polyethylene glycol molecules, sugars, phosphates, and/or other such molecules, where the molecule or molecules are not naturally attached to wild-type binding domain fusion proteins. A “derivative” of a polypeptide further includes those polypeptides that are “derived” from a reference polypeptide by having, for example, amino acid substitutions, deletions, or insertions relative to a reference polypeptide. Thus, a polypeptide may be “derived” from a wild-type polypeptide or from any other polypeptide. As used herein, a compound, including polypeptides, may also be “derived” from a particular source, for example from a particular organism, tissue type, or from a particular polypeptide, nucleic acid, or other compound that is present in a particular organism or a particular tissue type.

As used herein, the term “diagnosis” means detecting a disease or disorder or determining the stage or degree of a disease or disorder. Usually, a diagnosis of a disease or disorder is based on the evaluation of one or more factors and/or symptoms that are indicative of the disease. That is, a diagnosis can be made based on the presence, absence or amount of a factor which is indicative of presence or absence of the disease or condition. Each factor or symptom that is considered to be indicative for the diagnosis of a particular disease does not need be exclusively related to the particular disease; i.e. there may be differential diagnoses that can be inferred from a diagnostic factor or symptom. Likewise, there may be instances where a factor or symptom that is indicative of a particular disease is present in an individual that does not have the particular disease. The diagnostic methods may be used independently, or in combination with other diagnosing and/or staging methods known in the medical art for a particular disease or disorder.

As used herein, the phrase “difference of the level” refers to differences in the quantity of a particular marker, such as a nucleic acid or a protein, in a sample as compared to a control or reference level. For example, the quantity of a particular biomarker may be present at an elevated amount or at a decreased amount in samples of patients with a disease compared to a reference level. In some embodiments, a “difference of a level” may be a difference between the quantity of a particular biomarker present in a sample as compared to a control of at least about 1%, at least about 2%, at least about 3%, at least about 5%, at least about 10%, at least about 15%, at least about 20%, at least about 25%, at least about 30%, at least about 35%, at least about 40%, at least about 50%, at least about 60%, at least about 75%, at least about 80% or more. In some embodiments, a “difference of a level” may be a statistically significant difference between the quantity of a biomarker present in a sample as compared to a control. For example, a difference may be statistically significant if the measured level of the biomarker falls outside of about 1.0 standard deviations, about 1.5 standard deviations, about 2.0 standard deviations, or about 2.5 stand deviations of the mean of any control or reference group.

The term “control or reference standard” describes a material comprising none, or a normal, low, or high level of one of more of the marker (or biomarker) expression products of one or more the markers (or biomarkers) of the invention, such that the control or reference standard may serve as a comparator against which a sample can be compared.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

The terms “dysregulated” and “dysregulation” as used herein describes a decreased (down-regulated) or increased (up-regulated) level of expression of a miRNA present and detected in a sample obtained from subject as compared to the level of expression of that miRNA in a comparator sample, such as a comparator sample obtained from one or more normal, not-at-risk subjects, or from the same subject at a different time point. In some instances, the level of miRNA expression is compared with an average value obtained from more than one not-at-risk individuals. In other instances, the level of miRNA expression is compared with a miRNA level assessed in a sample obtained from one normal, not-at-risk subject.

By the phrase “determining the level of marker (or biomarker) expression” is meant an assessment of the degree of expression of a marker in a sample at the nucleic acid or protein level, using technology available to the skilled artisan to detect a sufficient portion of any marker expression product.

The terms “determining,” “measuring,” “assessing,” and “assaying” are used interchangeably and include both quantitative and qualitative measurement, and include determining if a characteristic, trait, or feature is present or not. Assessing may be relative or absolute. “Assessing the presence of” includes determining the amount of something present, as well as determining whether it is present or absent.

“Differentially increased expression” or “up regulation” refers to expression levels which are at least 10% or more, for example, 20%, 30%, 40%, or 50%, 60%, 70%, 80%, 90% higher or more, and/or 1.1 fold, 1.2 fold, 1.4 fold, 1.6 fold, 1.8 fold, 2.0 fold higher or more, and any and all whole or partial increments there between than a comparator.

“Differentially decreased expression” or “down regulation” refers to expression levels which are at least 10% or more, for example, 20%, 30%, 40%, or 50%, 60%, 70%, 80%, 90% lower or less, and/or 2.0 fold, 1.8 fold, 1.6 fold, 1.4 fold, 1.2 fold, 1.1 fold lower or less, and any and all whole or partial increments there between than a comparator.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

The term “expression” as used herein is defined as the transcription and/or translation of a particular nucleotide sequence.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′-ATTGCC-3′ and 5′-TATGGC-3′ share 50% homology.

As used herein, “homology” is used synonymously with “identity.”

“Inhibitors,” “activators,” and “modulators” of the markers are used to refer to activating, inhibitory, or modulating molecules identified using in vitro and in vivo assays of endometriosis biomarkers. Inhibitors are compounds that, e.g., bind to, partially or totally block activity, decrease, prevent, delay activation, inactivate, desensitize, or down regulate the activity or expression of endometriosis biomarkers. “Activators” are compounds that increase, open, activate, facilitate, enhance activation, sensitize, agonize, or up regulate activity of endometriosis biomarkers, e.g., agonists Inhibitors, activators, or modulators also include genetically modified versions of endometriosis biomarkers, e.g., versions with altered activity, as well as naturally occurring and synthetic ligands, antagonists, agonists, antibodies, peptides, cyclic peptides, nucleic acids, antisense molecules, ribozymes, RNAi, microRNA, and siRNA molecules, small organic molecules and the like. Such assays for inhibitors and activators include, e.g., expressing endometriosis biomarkers in vitro, in cells, or cell extracts, applying putative modulator compounds, and then determining the functional effects on activity, as described elsewhere herein.

As used herein, an “instructional material” includes a publication, a recording, a diagram, or any other medium of expression which can be used to communicate the usefulness of a compound, composition, vector, method or delivery system of the disclosure in the kit for effecting alleviation of the various diseases or disorders recited herein. Optionally, or alternately, the instructional material can describe one or more methods of alleviating the diseases or disorders in a cell or a tissue of a mammal. The instructional material of the kit of the disclosure can, for example, be affixed to a container which contains the identified compound, composition, vector, or delivery system of the disclosure or be shipped together with a container which contains the identified compound, composition, vector, or delivery system. Alternatively, the instructional material can be shipped separately from the container with the intention that the instructional material and the compound be used cooperatively by the recipient.

As used herein, “isolated” means altered or removed from the natural state through the actions, directly or indirectly, of a human being. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

“Measuring” or “measurement,” or alternatively “detecting” or “detection,” means assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's clinical parameters.

As used herein, “microRNA” or “miRNA” describes small non-coding RNA molecules, generally about 15 to about 50 nucleotides in length, preferably 17-23 nucleotides, which can play a role in regulating gene expression through, for example, a process termed RNA interference (RNAi). RNAi describes a phenomenon whereby the presence of an RNA sequence that is complementary or antisense to a sequence in a target gene messenger RNA (mRNA) results in inhibition of expression of the target gene. miRNAs are processed from hairpin precursors of about 70 or more nucleotides (pre-miRNA) which are derived from primary transcripts (pri-miRNA) through sequential cleavage by RNAse III enzymes. miRBase is a comprehensive microRNA database located at www.mirbase.org, incorporated by reference herein in its entirety for all purposes.

A “mutation,” as used herein, refers to a change in nucleic acid or polypeptide sequence relative to a reference sequence (which is preferably a naturally-occurring normal or “wild-type” sequence), and includes translocations, deletions, insertions, and substitutions/point mutations. A “mutant,” as used herein, refers to either a nucleic acid or protein comprising a mutation.

“Naturally occurring” as used herein describes a composition that can be found in nature as distinct from being artificially produced. For example, a nucleotide sequence present in an organism, which can be isolated from a source in nature and which has not been intentionally modified by a person, is naturally occurring.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand.” Sequences on the DNA strand which are located 5′ to a reference point on the DNA are referred to as “upstream sequences.” Sequences on the DNA strand which are 3′ to a reference point on the DNA are referred to as “downstream sequences.”

As used herein, “polynucleotide” includes cDNA, RNA, DNA/RNA hybrid, anti-sense RNA, siRNA, miRNA, genomic DNA, synthetic forms, and mixed polymers, both sense and antisense strands, and may be chemically or biochemically modified to contain non-natural or derivatized, synthetic, or semi-synthetic nucleotide bases. Also, included within the scope of the disclosure are alterations of a wild type or synthetic gene, including but not limited to deletion, insertion, substitution of one or more nucleotides, or fusion to other polynucleotide sequences.

As used herein, a “primer” for amplification is an oligonucleotide that specifically anneals to a target or marker nucleotide sequence. The 3′ nucleotide of the primer should be identical to the target or marker sequence at a corresponding nucleotide position for optimal primer extension by a polymerase. As used herein, a “forward primer” is a primer that anneals to the anti-sense strand of double stranded DNA (dsDNA). A “reverse primer” anneals to the sense-strand of dsDNA.

The term “recombinant DNA” as used herein is defined as DNA produced by joining pieces of DNA from different sources.

As used herein, the term “providing a prognosis” refers to providing a prediction of the probable course and outcome of endometriosis, including prediction of severity, duration, chances of recovery, etc. The methods can also be used to devise a suitable therapeutic plan, e.g., by indicating whether or not the condition is still at an early stage or if the condition has advanced to a stage where aggressive therapy would be ineffective.

A “reference level” of a biomarker means a level of the biomarker that is indicative of a particular disease state, phenotype, or lack thereof, as well as combinations of disease states, phenotypes, or lack thereof. A “positive” reference level of a biomarker means a level that is indicative of a particular disease state or phenotype. A “negative” reference level of a biomarker means a level that is indicative of a lack of a particular disease state or phenotype.

“Sample” or “biological sample” as used herein means a biological material isolated from an individual. The biological sample may contain any biological material suitable for detecting the desired biomarkers, and may comprise cellular and/or non-cellular material obtained from the individual.

“Standard control value” as used herein refers to a predetermined amount of a particular protein or nucleic acid that is detectable in a biological sample. The standard control value is suitable for the use of a method of the present disclosure, in order for comparing the amount of a protein or nucleic acid of interest that is present in a biological sample. An established sample serving as a standard control provides an average amount of the protein or nucleic acid of interest in the biological sample that is typical for an average, healthy person of reasonably matched background, e.g., gender, age, ethnicity, and medical history. A standard control value may vary depending on the protein or nucleic acid of interest and the nature of the sample (e.g., serum).

The terms “subject,” “patient,” “individual,” and the like are used interchangeably herein, and refer to any animal, or cells thereof whether in vitro or in situ, amenable to the methods described herein. In certain non-limiting embodiments, the patient, subject or individual is a human.

The terms “underexpress,” “underexpression,” “underexpressed,” or “down-regulated” interchangeably refer to a protein or nucleic acid that is transcribed or translated at a detectably lower level in a biological sample from a woman with endometriosis, in comparison to a biological sample from a woman without endometriosis. The term includes underexpression due to transcription, post transcriptional processing, translation, post-translational processing, cellular localization (e.g., organelle, cytoplasm, nucleus, cell surface), and RNA and protein stability, as compared to a control. Underexpression can be detected using conventional techniques for detecting mRNA (i.e., Q-PCR, RT-PCR, PCR, hybridization) or proteins (i.e., ELISA, immunohistochemical techniques). Underexpression can be 10%, 20%, 30%, 40%, 50%, 60%, 70%, 80%, 90% or less in comparison to a control. In certain instances, underexpression is 1-, 2-, 3-, 4-, 5-, 6-, 7-, 8-, 9-, 10-fold or more lower levels of transcription or translation in comparison to a control.

The terms “overexpress,” “overexpression,” “overexpressed,” or “up-regulated” interchangeably refer to a protein or nucleic acid (RNA) that is transcribed or translated at a detectably greater level, usually in a biological sample from a woman with endometriosis, in comparison to a biological sample from a woman without endometriosis. The term includes overexpression due to transcription, post transcriptional processing, translation, post-translational processing, cellular localization (e.g., organelle, cytoplasm, nucleus, cell surface), and RNA and protein stability, as compared to a cell from a woman without endometriosis. Overexpression can be detected using conventional techniques for detecting mRNA (i.e., Q-PCR, RT-PCR, PCR, hybridization) or proteins (i.e., ELISA, immunohistochemical techniques). Overexpression can be 10%, 20%, 30%, 40%, 50%, 60%, 70%, 80%, 90% or more in comparison to a cell from a woman without endometriosis. In certain instances, overexpression is 1-, 2-, 3-, 4-, 5-, 6-, 7-, 8-, 9-, 10-fold, or more higher levels of transcription or translation in comparison to a cell from a woman without endometriosis.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

As used herein, the terms “treat,” “ameliorate,” “treatment,” and “treating” are used interchangeably. These terms refer to an approach for obtaining beneficial or desired results including, but are not limited to, therapeutic benefit and/or a prophylactic benefit. Therapeutic benefit means eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder. For prophylactic benefit, treatment may be administered to a patient at risk of developing a particular disease, or to a patient reporting one or more of the physiological symptoms of a disease, even though a diagnosis of this disease may not have been made.

The term “or” as used herein and throughout the disclosure, generally means “and/or” unless the context dictates otherwise.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates to compositions and methods for treating endometriosis. For example, in certain aspects, the present inventions provide compositions for reducing lesion growth, and the like. The present invention is based in part upon the discovery that inhibition of miR451a decreased the size of established endometrial lesions and decreased expression levels of genes that have a role in the pathophysiology of endometriosis.

In one aspect, the present invention relates to a composition for treating and preventing endometriosis. In one embodiment, the composition comprises an inhibitor of miR451a. A miR451a inhibitor may be any type of compound, including but not limited to, a polypeptide, a nucleic acid, an aptamer, an anti-miR, antagomiR, a miR sponge, a silencing RNA (siRNA), a short hairpin RNA (shRNA), a morpholino, a piwi-interacting RNA (piRNA), a repeat associated small interfering RNA (rasiRNAs), and a small molecule, or combinations thereof.

In certain embodiments, the agent comprises an antisense oligonucleotide to miR451a. For example, in one embodiment, the agent comprises a sequence at least 90% homologous to AAACCGUUACCAUUACUGAGUU (SEQ ID NO:1).

In one embodiment, the present invention provides a method for treating or preventing endometriosis in a subject. For example, in one embodiment, the method comprises administering to the subject an inhibitor of miR451a. For example, in one embodiment, the method comprises administering to the subject an effective amount of an agent that decreases the expression or activity of miR451a. In one embodiment, the method comprises administering to the subject one or more antisense oligonucleotide molecule targeting miR451a, including but not limited to an oligonucleotide comprising a sequence at least 90% homologous to

(SEQ ID NO: 1)
AAACCGUUACCAUUACUGAGUU.

Compositions

In one embodiment, the composition of the present invention comprises an inhibitor of miR451a. For example, in one embodiment, the inhibitor of miR451a reduces the expression, activity, or both of miR451a. In certain embodiments, the inhibitor comprises a polypeptide, a nucleic acid, an aptamer, an anti-miR, antagomiR, a miR sponge, a silencing RNA (siRNA), a short hairpin RNA (shRNA), a morpholino, a piwi-interacting RNA (piRNA), a repeat associated small interfering RNA (rasiRNAs), and a small molecule, or combinations thereof that decreases the level or expression of miR451a, activity of miR451a, or a combination thereof. In certain embodiments, the inhibitor comprises an antisense oligonucleotide targeting miR451a. In one embodiment, the inhibitor comprises an oligonucleotide having a sequence of SEQ ID NO:1 or a mimic of an oligonucleotide having a sequence of SEQ ID NO:1.

In one embodiment, the present invention provides a composition for treating or preventing endometriosis in a subject. In one embodiment, the composition inhibits lesion growth. In certain embodiments, the composition decreases the expression, activity, or both of miR451a in a cell of the subject.

It will be understood by one skilled in the art, based upon the disclosure provided herein, that modulating a miRNA encompasses modulating the level or activity of a miRNA including, but not limited to, modulating the transcription, processing, nuclear export, splicing, degradation, binding activity, or combinations thereof. Thus, decreasing or inhibiting the level or activity of a miRNA includes, but is not limited to, decreasing transcription, processing, nuclear export, splicing, or binding activity, or binding activity or increasing degradation or combinations thereof; and it also includes modulating the level of any nucleic acid or protein that modulates the miRNA level or activity.

Small Molecule

When the inhibitor is a small molecule, a small molecule may be obtained using standard methods known to the skilled artisan. Such methods include chemical organic synthesis or biological means. Biological means include purification from a biological source, recombinant synthesis and in vitro translation systems, using methods well known in the art. In one embodiment, a small molecule inhibitor of the invention comprises an organic molecule, inorganic molecule, biomolecule, synthetic molecule, and the like.

Combinatorial libraries of molecularly diverse chemical compounds potentially useful in treating a variety of diseases and conditions are well known in the art as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

The small molecule and small molecule compounds described herein may be present as salts even if salts are not depicted and it is understood that the invention embraces all salts and solvates of the compounds depicted here, as well as the non-salt and non-solvate form of the compounds, as is well understood by the skilled artisan. In some embodiments, the salts of the compounds of the invention are pharmaceutically acceptable salts.

Where tautomeric forms may be present for any of the compounds described herein, each and every tautomeric form is intended to be included in the present invention, even though only one or some of the tautomeric forms may be explicitly depicted. For example, when a 2-hydroxypyridyl moiety is depicted, the corresponding 2-pyridone tautomer is also intended.

The invention also includes any or all of the stereochemical forms, including any enantiomeric or diasteriomeric forms of the compounds described. The recitation of the structure or name herein is intended to embrace all possible stereoisomers of compounds depicted. All forms of the compounds are also embraced by the invention, such as crystalline or non-crystalline forms of the compounds. Compositions comprising a compound of the invention are also intended, such as a composition of substantially pure compound, including a specific stereochemical form thereof, or a composition comprising mixtures of compounds of the invention in any ratio, including two or more stereochemical forms, such as in a racemic or non-racemic mixture.

In one embodiment, the small molecule compound of the invention comprises an analog or derivative of a compound described herein.

In one embodiment, the small molecules described herein are candidates for derivatization. As such, in certain instances, the analogs of the small molecules described herein that have modulated potency, selectivity, and solubility are included herein and provide useful leads for drug discovery and drug development. Thus, in certain instances, during optimization new analogs are designed considering issues of drug delivery, metabolism, novelty, and safety.

In some instances, small molecule inhibitors described herein are derivatized/analoged as is well known in the art of combinatorial and medicinal chemistry. The analogs or derivatives can be prepared by adding and/or substituting functional groups at various locations. As such, the small molecules described herein can be converted into derivatives/analogs using well known chemical synthesis procedures. For example, all of the hydrogen atoms or substituents can be selectively modified to generate new analogs. Also, the linking atoms or groups can be modified into longer or shorter linkers with carbon backbones or hetero atoms. Also, the ring groups can be changed so as to have a different number of atoms in the ring and/or to include hetero atoms. Moreover, aromatics can be converted to cyclic rings, and vice versa. For example, the rings may be from 5-7 atoms, and may be homocycles or heterocycles.

As used herein, the term “analog”, “analogue,” or “derivative” is meant to refer to a chemical compound or molecule made from a parent compound or molecule by one or more chemical reactions. As such, an analog can be a structure having a structure similar to that of the small molecule compounds described herein or can be based on a scaffold of a small molecule compound described herein, but differing from it in respect to certain components or structural makeup, which may have a similar or opposite action metabolically. An analog or derivative of any of a small molecule compound in accordance with the present invention can be used to decrease the expression of miR451a, the activity of miR451a, or both.

In one embodiment, the small molecule compounds described herein can independently be derivatized/analoged by modifying hydrogen groups independently from each other into other substituents. That is, each atom on each molecule can be independently modified with respect to the other atoms on the same molecule. Any traditional modification for producing a derivative/analog can be used. For example, the atoms and substituents can be independently comprised of hydrogen, an alkyl, aliphatic, straight chain aliphatic, aliphatic having a chain hetero atom, branched aliphatic, substituted aliphatic, cyclic aliphatic, heterocyclic aliphatic having one or more hetero atoms, aromatic, heteroaromatic, polyaromatic, polyamino acids, peptides, polypeptides, combinations thereof, halogens, halo-substituted aliphatics, and the like. Additionally, any ring group on a compound can be derivatized to increase and/or decrease ring size as well as change the backbone atoms to carbon atoms or hetero atoms.

Nucleic Acids

In certain embodiments, the composition comprises a modulator of miR451a described herein. For example, in certain embodiments, the composition comprises an agent that decreases the expression, level or activity of miR451a. In one embodiment, the agent comprises an antisense nucleic acid molecule that targets miR451a. In certain embodiments, the composition comprises a sequence at least 90% identical to SEQ ID NO:1.

miRNAs are small non-coding RNA molecules that are capable of causing post-transcriptional silencing of specific genes in cells by the inhibition of translation or through degradation of the targeted mRNA. A miRNA can be completely complementary or can have a region of non-complementarity with a target nucleic acid, consequently resulting in a “bulge” at the region of non-complementarity. A miRNA can inhibit gene expression by repressing translation, such as when the miRNA is not completely complementary to the target nucleic acid, or by causing target RNA degradation, which is believed to occur only when the miRNA binds its target with perfect complementarity. The disclosure also can include double-stranded precursors of miRNA. A miRNA or pri-miRNA can be 18-100 nucleotides in length. In one embodiment, the miRNA or pri-miRNA is about 18-80 nucleotides in length. Mature miRNAs can have a length of 19-30 nucleotides. In one embodiment, the mature miRNAs can have a length of about 21-25 nucleotides. In one embodiment, the mature miRNAs can have a length of about 21, 22, 23, 24, or 25 nucleotides. miRNA precursors typically have a length of about 70-100 nucleotides and have a hairpin conformation. miRNAs are generated in vivo from pre-miRNAs by the enzymes Dicer and Drosha, which specifically process long pre-miRNA into functional miRNA. The hairpin or mature microRNAs, or pri-microRNA agents featured in the disclosure can be synthesized in vivo by a cell-based system or in vitro by chemical synthesis.

While, in specific instances, the description may refer to miRNA species having a 5p or 3p notation, the present invention encompasses the use of both the 5p and 3p versions of each miRNA species. Sequences of the miRNA family members are publicly available from miRbase.

In various embodiments, agent comprises an oligonucleotide that contains the antisense nucleotide sequence of miR451a. In certain embodiments, the oligonucleotide comprises the antisense nucleotide sequence of miR451a in a pre-microRNA, mature or hairpin form. In other embodiments, a combination of oligonucleotides comprising an antisense nucleotide sequence of miR451a, any pre-miRNA, any fragment, or any combination thereof is envisioned.

Antisense oligonucleotides can be synthesized to include a modification that imparts a desired characteristic. For example, the modification can improve stability, hybridization thermodynamics with a target nucleic acid, targeting to a particular tissue or cell-type, or cell permeability, e.g., by an endocytosis-dependent or -independent mechanism.

Modifications can also increase sequence specificity, and consequently decrease off-site targeting. Methods of synthesis and chemical modifications are described in greater detail below. If desired, miRNA molecules may be modified to stabilize the miRNAs against degradation, to enhance half-life, or to otherwise improve efficacy. Desirable modifications are described, for example, in U.S. Patent Publication Nos. 20070213292, 20060287260, 20060035254. 20060008822. and 2005028824, each of which is hereby incorporated by reference in its entirety. For increased nuclease resistance and/or binding affinity to the target, the single-stranded oligonucleotide agents featured in the disclosure can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleotide modifications can also increase binding affinity to the target. The inclusion of pyranose sugars in the oligonucleotide backbone can also decrease endonucleolytic cleavage. A oligonucleotide can be further modified by including a 3′ cationic group, or by inverting the nucleoside at the 3′-terminus with a 3-3′ linkage. In another alternative, the 3 ‘-terminus can be blocked with an aminoalkyl group. Other 3’ conjugates can inhibit 3′-5′ exonucleolytic cleavage. While not being bound by theory, a 3′ may inhibit exonucleolytic cleavage by sterically blocking the exonuclease from binding to the 3′ end of the oligonucleotide. Even small alkyl chains, aryl groups, or heterocyclic conjugates or modified sugars (D-ribose, deoxyribose, glucose etc.) can block 3′-5′-exonucleases.

In one embodiment, the miRNA includes a 2′-modified oligonucleotide containing oligodeoxynucleotide gaps with some or all internucleotide linkages modified to phosphorothioates for nuclease resistance. The presence of methylphosphonate modifications increases the affinity of the oligonucleotide for its target RNA and thus reduces the IC5Q. This modification also increases the nuclease resistance of the modified oligonucleotide. It is understood that the methods and reagents of the present disclosure may be used in conjunction with any technologies that may be developed to enhance the stability or efficacy of an inhibitory nucleic acid molecule.

In one embodiment, antisense oligonucleotide molecules include nucleotide oligomers containing modified backbones or non-natural internucleoside linkages. Oligomers having modified backbones include those that retain a phosphorus atom in the backbone and those that do not have a phosphorus atom in the backbone. For the purposes of this disclosure, modified oligonucleotides that do not have a phosphorus atom in their internucleoside backbone are also considered to be nucleotide oligomers. Nucleotide oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates, thionophosphoramidates, thionoalkylphosphonates, thionoalkylphosphotriest-ers, and boranophosphates. Various salts, mixed salts and free acid forms are also included. Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Nucleotide oligomers having modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyl eneimino and methylenehydrazino backbones; sulfonate and sulfonamide backbones; amide backbones; and others having mixed N, O, S and CH₂ component parts. Representative United States patents that teach the preparation of the above oligonucleotides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference. Nucleotide oligomers may also contain one or more substituted sugar moieties. Such modifications include 2′-O-methyl and 2′-methoxyethoxy modifications. Another desirable modification is 2′-dimethylaminooxyethoxy, 2′-aminopropoxy and 2′-fluoro. Similar modifications may also be made at other positions on an oligonucleotide or other nucleotide oligomer, particularly the 3′ position of the sugar on the 3′ terminal nucleotide. Nucleotide oligomers may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

In other nucleotide oligomers, both the sugar and the internucleoside linkage, i.e., the backbone, are replaced with groups. Methods for making and using these nucleotide oligomers are described, for example, in “Peptide Nucleic Acids (PNA): Protocols and Applications” Ed. P. E. Nielsen, Horizon Press, Norfolk, United Kingdom, 1999. Representative United States patents that teach the preparation of PNAs include, but are not limited to, U.S. Pat. Nos. 5,539,082; 5,714,331; and 5,719,262, each of which is herein incorporated by reference. Further teaching of PNA compounds can be found in Nielsen et al, Science, 1991, 254, 1497-1500.

In other embodiments, a single stranded modified nucleic acid molecule (e.g., a nucleic acid molecule comprising a phosphorothioate backbone and 2′-OMe sugar modifications is conjugated to cholesterol.

An inhibitor as described herein, which targets miR451a, which may be in the mature or hairpin form, may be provided as a naked oligonucleotide that is capable of entering a cell. In some cases, it may be desirable to utilize a formulation that aids in the delivery of a miRNA or other nucleotide oligomer to cells (see, e.g., U.S. Pat. Nos. 5,656,611, 5,753,613, 5,785,992, 6,120,798, 6,221,959, 6,346,613, and 6,353,055, each of which is hereby incorporated by reference).

In some examples, the inhibitor composition is at least partially crystalline, uniformly crystalline, and/or anhydrous (e.g., less than 80, 50, 30, 20, or 10% water). In another example, the inhibitor composition is in an aqueous phase, e.g., in a solution that includes water. The aqueous phase or the crystalline compositions can be incorporated into a delivery vehicle, e.g., a liposome (particularly for the aqueous phase), or a particle (e.g., a microparticle as can be appropriate for a crystalline composition). Generally, the inhibitor composition is formulated in a manner that is compatible with the intended method of administration. An inhibitor of the invention can be formulated in combination with another agent, e.g., another therapeutic agent or an agent that stabilizes an oligonucleotide agent, e.g., a protein that complexes with the oligonucleotide agent. Still other agents include chelators, e.g., EDTA (e.g., to remove divalent cations such as Mg), salts, and RNAse inhibitors (e.g., a broad specificity RNAse inhibitor). In one embodiment, the miRNA composition includes another miRNA, e.g., a second miRNA composition (e.g., a microRNA that is distinct from the first). Still other preparations can include at least three, five, ten, twenty, fifty, or a hundred or more different oligonucleotide species.

In one embodiment, the antisense oligonucleotide of the invention targets an endogenous miR451a or a miR451a precursor nucleobase sequence. An oligonucleotide selected for inclusion in a composition of the present invention may be one of a number of lengths. Such an oligonucleotide can be from 7 to 100 linked nucleosides in length. For example, an antisense oligonucleotide to miR451a may be from 7 to 30 linked nucleosides in length. An antisense oligonucleotide to a miR451a precursor may be up to 100 linked nucleosides in length. In certain embodiments, an oligonucleotide comprises 7 to 30 linked nucleosides. In certain embodiments, an oligonucleotide comprises 7, 8, 9, 10, 11, 12, 13, 14, 15, 16, 17, 18, 19, 20, 21, 22, 23, 24, 25, 26, 28, 29, or 30 linked nucleotides. In certain embodiments, an oligonucleotide comprises 19 to 23 linked nucleosides. In certain embodiments, an oligonucleotide is from 40 up to 50, 60, 70, 80, 90, or 100 linked nucleosides in length.

In certain embodiments, an oligonucleotide has a sequence that is antisense to miR451a or a precursor thereof. Nucleobase sequences of mature miRNAs and their corresponding stem-loop sequences described herein are the sequences found in miRBase, an online searchable database of miRNA sequences and annotation. Entries in the miRBase Sequence database represent a predicted hairpin portion of a miRNA transcript (the stem-loop), with information on the location and sequence of the mature miRNA sequence. The miRNA stem-loop sequences in the database are not strictly precursor miRNAs (pre-miRNAs), and may in some instances include the pre-miRNA and some flanking sequence from the presumed primary transcript. The miRNA nucleobase sequences described herein encompass any version of the miRNA, including the sequences described in Release 10.0 of the miRBase sequence database and sequences described in any earlier Release of the miRBase sequence database. A sequence database release may result in the re-naming of certain miRNAs. A sequence database release may result in a variation of a mature miRNA sequence. The compositions of the present invention encompass oligomeric compound comprising oligonucleotides having a certain identity to any nucleobase sequence version of a miRNAs described herein.

In certain embodiments, an oligonucleotide has a nucleobase sequence at least 60%, 65%, 70%, 75%, 80%, 85%, 90%, 95%, 97%, 98% or 99% identical to the complement of the miRNA over a region of 7, 8, 9, 10, 11, 12, 13, 14, 15, 16, 17, 18, 19, 20, 21, 22, 23, 24, 25, 26, 27, 28, 29, or 30 nucleobases. Accordingly, in certain embodiments the nucleobase sequence of an oligonucleotide may have one or more non-identical nucleobases with respect to the miRNA. I

In certain embodiments, the composition comprises a nucleic acid molecule encoding an antisense oligonucleotide, variant thereof, or fragment thereof. For example, the composition may comprise a viral vector, plasmid, cosmid, or other expression vector suitable for expressing the antisense oligonucleotide, variant thereof, or fragment thereof in a desired mammalian cell or tissue.

In other related aspects, the invention includes an isolated nucleic acid. In some instances, the inhibitor is an siRNA, antisense molecule, or CRISPR guide RNA, which targets and inhibits miR451a. In one embodiment, the nucleic acid comprises a promoter/regulatory sequence such that the nucleic acid is capable of directing expression of the nucleic acid. Thus, the invention encompasses expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York), and in Ausubel et al. (2008, Current Protocols in Molecular Biology, John Wiley & Sons, New York) and as described elsewhere herein. In one embodiment, siRNA is used to decrease the level of miR451a. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. In some aspects, the level of miR451a can be decreased by increasing the level or activity of a protein or nucleic acid that degrades or inhibits miR451a. Therefore, the present invention also includes methods of modulating levels of one or more miR451a regulator.

In another aspect, the invention includes a vector comprising an siRNA or antisense polynucleotide. In one embodiment, the siRNA or antisense polynucleotide is capable of inhibiting the expression of a target polypeptide. In one embodiment, the siRNA or antisense polynucleotide is capable of increasing the expression of a target miRNA. The incorporation of a desired polynucleotide into a vector and the choice of vectors is well-known in the art as described in, for example, Sambrook et al., supra, and Ausubel et al., supra, and elsewhere herein.

In certain embodiments, the expression vectors described herein encode a short hairpin RNA (shRNA). shRNA are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleaves the shRNA to form siRNA.

The siRNA, shRNA, or antisense polynucleotide can be cloned into a number of types of vectors as described elsewhere herein. For expression of the siRNA or antisense polynucleotide, at least one module in each promoter functions to position the start site for RNA synthesis.

In order to assess the expression of the siRNA, shRNA, or antisense polynucleotide, the expression vector to be introduced into a cell can also contain either a selectable marker gene or a reporter gene or both to facilitate identification and selection of expressing cells from the population of cells sought to be transfected or infected using a viral vector. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neomycin resistance and the like.

Therefore, in another aspect, the invention relates to a vector, comprising the nucleotide sequence of the invention or the construct of the invention. The choice of the vector will depend on the host cell in which it is to be subsequently introduced. In a particular embodiment, the vector of the invention is an expression vector. Suitable host cells include a wide variety of prokaryotic and eukaryotic host cells. In specific embodiments, the expression vector is selected from the group consisting of a viral vector, a bacterial vector and a mammalian cell vector. Prokaryote- and/or eukaryote-vector based systems can be employed for use with the present invention to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al., and in Ausubel et al., and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

Vectors suitable for the insertion of the polynucleotides are vectors derived from expression vectors in prokaryotes such as pUC18, pUC19, Bluescript and the derivatives thereof, mp18, mp19, pBR322, pMB9, ColE1, pCR1, RP4, phages and “shuttle” vectors such as pSA3 and pAT28, expression vectors in yeasts such as vectors of the type of 2 micron plasmids, integration plasmids, YEP vectors, centromere plasmids and the like, expression vectors in insect cells such as vectors of the pAC series and of the pVL, expression vectors in plants such as pIBI, pEarleyGate, pAVA, pCAMBIA, pGSA, pGWB, pMDC, pMY, pORE series and the like, and expression vectors in eukaryotic cells based on viral vectors (adenoviruses, viruses associated to adenoviruses such as retroviruses and, particularly, lentiviruses) as well as non-viral vectors such as pSilencer 4.1-CMV (Ambion), pcDNA3, pcDNA3.1/hyg, pHMCV/Zeo, pCR3.1, pEFI/His, pIND/GS, pRc/HCMV2, pSV40/Zeo2, pTRACER-HCMV, pUB6N5-His, pVAX1, pZeoSV2, pCI, pSVL and PKSV-10, pBPV-1, pML2d and pTDT1.

By way of illustration, the vector in which the nucleic acid sequence is introduced can be a plasmid which is or is not integrated in the genome of a host cell when it is introduced in the cell. Illustrative, non-limiting examples of vectors in which the nucleotide sequence of the invention or the gene construct of the invention can be inserted include a tet-on inducible vector for expression in eukaryote cells.

The vector may be obtained by conventional methods known by persons skilled in the art (Sambrook et al.). In a particular embodiment, the vector is a vector useful for transforming animal cells.

In one embodiment, the recombinant expression vectors may also contain nucleic acid molecules which encode a peptide or peptidomimetic of invention, described elsewhere herein.

Additional promoter elements, i.e., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either co-operatively or independently to activate transcription.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

A promoter sequence exemplified in the experimental examples presented herein is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, Moloney virus promoter, the avian leukemia virus promoter, Epstein-Barr virus immediate early promoter, Rous sarcoma virus promoter, as well as human gene promoters such as, but not limited to, the actin promoter, the myosin promoter, the hemoglobin promoter, and the muscle creatine promoter. Further, the invention should not be limited to the use of constitutive promoters. Inducible promoters are also contemplated as part of the invention. The use of an inducible promoter in the invention provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. Further, the invention includes the use of a tissue specific promoter, which promoter is active only in a desired tissue (e.g., skin). Tissue specific promoters are well known in the art and include, but are not limited to, the keratin 14 promoter and the fascin promoter sequences.

In a particular embodiment, the expression of the nucleic acid is externally controlled. In a more particular embodiment, the expression is externally controlled using the doxycycline Tet-On system.

The recombinant expression vectors may also contain a selectable marker gene which facilitates the selection of transformed or transfected host cells. Suitable selectable marker genes are genes encoding proteins such as G418 and hygromycin which confer resistance to certain drugs, β-galactosidase, chloramphenicol acetyltransferase, firefly luciferase, or an immunoglobulin or portion thereof such as the Fc portion of an immunoglobulin such as IgG. The selectable markers may be introduced on a separate vector from the nucleic acid of interest.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000 FEBS Lett. 479:79-82). Suitable expression systems are well known and may be prepared using well known techniques or obtained commercially. Internal deletion constructs may be generated using unique internal restriction sites or by partial digestion of non-unique restriction sites. Constructs may then be transfected into cells that display high levels of siRNA polynucleotide and/or polypeptide expression. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Recombinant expression vectors may be introduced into host cells to produce a recombinant cell. The cells can be prokaryotic or eukaryotic. The vector of the invention can be used to transform eukaryotic cells such as yeast cells, Saccharomyces cerevisiae, or mammal cells for example epithelial kidney 293 cells or U2OS cells, or prokaryotic cells such as bacteria, Escherichia coli or Bacillus subtilis, for example. Nucleic acid can be introduced into a cell using conventional techniques such as calcium phosphate or calcium chloride co-precipitation, DEAE-dextran-mediated transfection, lipofectin, electroporation or microinjection. Suitable methods for transforming and transfecting host cells may be found in Sambrook et al. (Molecular Cloning: A Laboratory Manual, 2nd Edition, Cold Spring Harbor Laboratory press (1989)), and other laboratory textbooks.

Following the generation of the siRNA polynucleotide, a skilled artisan will understand that the siRNA polynucleotide will have certain characteristics that can be modified to improve the siRNA as a therapeutic compound. Therefore, the siRNA polynucleotide may be further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrwal et al., 1987 Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl-methyl-, thio- and other modified forms of adenine, cytidine, guanine, thymine, and uridine.

In one embodiment of the invention, an antisense nucleic acid sequence which is expressed by a plasmid vector is used to decrease the level of miR451a. The antisense expressing vector is used to transfect a mammalian cell or the mammal itself, thereby causing decreasing endogenous levels of miR451a.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. In one embodiment, an antisense oligomer comprises between about 10 to about 30 nucleotides. In one embodiment, an antisense oligomer comprises about 15 nucleotides. Antisense oligomers comprising 10-30 nucleotides are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

Compositions and methods for the synthesis and expression of antisense nucleic acids are as described elsewhere herein.

Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053). Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). A major advantage of this approach is the fact that ribozymes are sequence-specific.

There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules.

In one embodiment of the invention, a ribozyme is used to decrease the level of miR451a. Ribozymes useful for inhibiting miR451a may be designed by incorporating target sequences into the basic ribozyme structure which are complementary, for example, to the miR451a sequence. Ribozymes which decrease or inhibit miR451a, may be synthesized using commercially available reagents (Applied Biosystems, Inc., Foster City, Calif.) or they may be genetically expressed from DNA encoding them.

Small Molecule Inhibitors of miRs

Small molecules, including inorganic and organic chemicals, peptides and peptoids, have been reported as small molecule drugs targeting specific miRs (SMIRs). Therefore, in one embodiment, the invention relates to compositions comprising a small molecule inhibitor of a miR of the invention. In one embodiment, a small molecule of the invention will have specific binding affinity to a mature miR or a pre-miR. In one embodiment, the composition comprises a SMIR targeting miR451a.

Anti-miR Oligonucleotides

Anti-miR oligonucleotides (AMOs) are generally single-stranded, chemically modified DNA-like molecules that are designed to be complementary to and inhibit a selected miR. In one embodiment, the composition comprises an AMO targeting miR451a. miRs are incorporated into ribonucleoprotein particles (miRNPs) which predominantly act as translational repressors. AMOs are single stranded anti-microRNA molecules which are capable of inhibiting miRNP activity.

In one embodiment, the AMO is a modified oligonucleotides. In one embodiment, the phosphate backbone of the AMO is modified. A modification of an AMO may include, but is not limited to, a LNA modification, a morpholino modification and a chemical modification. LNA is a bicyclic RNA analogue in which the ribose is locked in a C3′-endo conformation by introduction of a 2′-0,4′-C methylene bridge. Morpholinos are uncharged, inherently resistant to degradation by nucleases. A representative United States patent application that teaches the preparation of such AMOs is published U.S. Application No. 20050182005A1 which is hereby incorporated by reference in its entirety.

In one embodiment, the invention includes a vector for expression of an anti-miR of the invention. In one embodiment, the vector is an expression vector designed to mediate the delivery of small RNAs in mammalian cells. In one embodiment, the expression vector is designed to stably express an anti-miR of the invention. The anti-miR oligonucleotide can be cloned into a number of types of vectors, including but not limited to lentiviral expression vectors.

In order to assess the expression of the anti-miR oligonucleotide, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neomycin resistance and the like.

Alternatively, anti-miR oligonucleotides of the invention may be made synthetically and then provided to the cell. Compositions and methods for the synthesis and administration of anti-miR oligonucleotides are as described elsewhere herein.

miR Sponges

In one embodiment, an inhibitor of miR451a may be in the form of a miR sponge. miR sponges are RNA transcripts produced from transgenes expressed in cells that contain multiple binding sites for a target miR. In one embodiment, a miR sponge may be expressed in a cell using an expression vector and administered using gene therapy methods.

Polypeptides

In other related aspects, the invention includes an isolated peptide that inhibits miR451a. For example, in one embodiment, the peptide of the invention inhibits miR451a directly by binding to, competing with, or acting as a transdominant negative mutant of a miR451a thereby inhibiting the normal functional activity of miR451a.

The variants of the polypeptides according to the present invention may be (i) one in which one or more of the amino acid residues are substituted with a conserved or non-conserved amino acid and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

The polypeptides of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The polypeptides of the invention may include unnatural amino acids formed by post-translational modification or by introducing unnatural amino acids during translation. A variety of approaches are available for introducing unnatural amino acids during protein translation. By way of example, special tRNAs, such as tRNAs which have suppressor properties, suppressor tRNAs, have been used in the process of site-directed non-native amino acid replacement (SNAAR). In SNAAR, a unique codon is required on the mRNA and the suppressor tRNA, acting to target a non-native amino acid to a unique site during the protein synthesis (described in WO90/05785). However, the suppressor tRNA must not be recognizable by the aminoacyl tRNA synthetases present in the protein translation system. In certain cases, a non-native amino acid can be formed after the tRNA molecule is aminoacylated using chemical reactions which specifically modify the native amino acid and do not significantly alter the functional activity of the aminoacylated tRNA. These reactions are referred to as post-aminoacylation modifications. For example, the epsilon-amino group of the lysine linked to its cognate tRNA (tRNALys), could be modified with an amine specific photoaffinity label.

A peptide of the invention may be conjugated with other molecules, such as proteins, to prepare fusion proteins. This may be accomplished, for example, by the synthesis of N-terminal or C-terminal fusion proteins provided that the resulting fusion protein retains the functionality of the peptide.

Cyclic derivatives of the peptides or chimeric proteins of the invention are also part of the present invention. Cyclization may allow the peptide or chimeric protein to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The components that form the bonds may be side chains of amino acids, non-amino acid components or a combination of the two. In an embodiment of the invention, cyclic peptides may comprise a beta-turn in the right position. Beta-turns may be introduced into the peptides of the invention by adding the amino acids Pro-Gly at the right position.

In other embodiments, the subject peptide therapeutics are peptidomimetics of the peptides. Peptidomimetics are compounds based on, or derived from, peptides and proteins. The peptidomimetics of the present invention typically can be obtained by structural modification of a known peptide sequence using unnatural amino acids, conformational restraints, isosteric replacement, and the like. The subject peptidomimetics constitute the continuum of structural space between peptides and non-peptide synthetic structures; peptidomimetics may be useful, therefore, in delineating pharmacophores and in helping to translate peptides into nonpeptide compounds with the activity of the parent peptides.

Moreover, as is apparent from the present disclosure, mimetopes of the subject peptide can be provided. Such peptidomimetics can have such attributes as being non-hydrolyzable (e.g., increased stability against proteases or other physiological conditions which degrade the corresponding peptide), increased specificity and/or potency, and increased cell permeability for intracellular localization of the peptidomimetic.

Peptides of the invention may be developed using a biological expression system. The use of these systems allows the production of large libraries of random peptide sequences and the screening of these libraries for peptide sequences that bind to particular proteins. Libraries may be produced by cloning synthetic DNA that encodes random peptide sequences into appropriate expression vectors. (see Christian et al 1992, J. Mol. Biol. 227:711; Devlin et al, 1990 Science 249:404; Cwirla et al 1990, Proc. Natl. Acad, Sci. USA, 87:6378). Libraries may also be constructed by concurrent synthesis of overlapping peptides (see U.S. Pat. No. 4,708,871).

The peptides and chimeric proteins of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

Antibodies and peptides may be modified using ordinary molecular biological techniques to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The polypeptides useful in the invention may further be conjugated to non-amino acid moieties that are useful in their application. In particular, moieties that improve the stability, biological half-life, water solubility, and immunologic characteristics of the peptide are useful. A non-limiting example of such a moiety is polyethylene glycol (PEG).

Antibodies

The invention also contemplates an antibody, or antibody fragment, specific for a protein which activates miR451a thereby inhibiting the normal functional activity of miR451a. That is, the antibody can inhibit a protein which itself activates or increases miR451a expression to teat or prevent endometriosis.

Methods of making and using antibodies are well known in the art. For example, polyclonal antibodies useful in the present invention are generated by immunizing rabbits according to standard immunological techniques well-known in the art (see, e.g., Greenfield et al., 2014, Antibodies, A Laboratory Manual, Cold Spring Harbor, N.Y.). Such techniques include immunizing an animal with a chimeric protein comprising a portion of another protein such as a maltose binding protein or glutathione (GSH) tag polypeptide portion, and/or a moiety such that the antigenic protein of interest is rendered immunogenic (e.g., an antigen of interest conjugated with keyhole limpet hemocyanin, KLH) and a portion comprising the respective antigenic protein amino acid residues. The chimeric proteins are produced by cloning the appropriate nucleic acids encoding the marker protein into a plasmid vector suitable for this purpose, such as but not limited to, pMAL-2 or pCMX.

One skilled in the art would appreciate, based upon the disclosure provided herein, that the antibody can specifically bind with any portion of the antigen and the full-length protein can be used to generate antibodies specific therefor. However, the present invention is not limited to using the full-length protein as an immunogen. Rather, the present invention includes using an immunogenic portion of the protein to produce an antibody that specifically binds with a specific antigen. That is, the invention includes immunizing an animal using an immunogenic portion, or antigenic determinant, of the antigen.

Once armed with the sequence of a specific antigen of interest and the detailed analysis localizing the various conserved and non-conserved domains of the protein, the skilled artisan would understand, based upon the disclosure provided herein, how to obtain antibodies specific for the various portions of the antigen using methods well-known in the art or to be developed.

The skilled artisan would appreciate, based upon the disclosure provided herein, that that present invention includes use of a single antibody recognizing a single antigenic epitope but that the invention is not limited to use of a single antibody. Instead, the invention encompasses use of at least one antibody where the antibodies can be directed to the same or different antigenic protein epitopes.

The generation of polyclonal antibodies is accomplished by inoculating the desired animal with the antigen and isolating antibodies which specifically bind the antigen therefrom using standard antibody production methods.

Monoclonal antibodies directed against full length or peptide fragments of a protein or peptide may be prepared using any well-known monoclonal antibody preparation procedures. Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art. Further, the antibody of the invention may be “humanized” using methods of humanizing antibodies well-known in the art or to be developed.

The present invention also includes the use of humanized antibodies specifically reactive with epitopes of an antigen of interest. The humanized antibodies of the invention have a human framework and have one or more complementarity determining regions (CDRs) from an antibody, typically a mouse antibody, specifically reactive with an antigen of interest.

The invention also includes functional equivalents of the antibodies described herein. Functional equivalents have binding characteristics comparable to those of the antibodies, and include, for example, hybridized and single chain antibodies, as well as fragments thereof.

Functional equivalents include polypeptides with amino acid sequences substantially the same as the amino acid sequence of the variable or hypervariable regions of the antibodies. “Substantially the same” amino acid sequence is defined herein as a sequence with at least 70%, at least about 80%, at least about 90%, at least about 95%, or at least 99% homology to another amino acid sequence (or any integer in between 70 and 99), as determined by the FASTA search method. Chimeric or other hybrid antibodies have constant regions derived substantially or exclusively from human antibody constant regions and variable regions derived substantially or exclusively from the sequence of the variable region of a monoclonal antibody from each stable hybridoma.

Single chain antibodies (scFv) or Fv fragments are polypeptides that consist of the variable region of the heavy chain of the antibody linked to the variable region of the light chain, with or without an interconnecting linker. Thus, the Fv comprises an antibody combining site.

Functional equivalents of the antibodies of the invention further include fragments of antibodies that have the same, or substantially the same, binding characteristics to those of the whole antibody. Such fragments may contain one or both Fab fragments or the F(ab′)2 fragment. The antibody fragments contain all six complement determining regions of the whole antibody, although fragments containing fewer than all of such regions, such as three, four or five complement determining regions, are also functional. The functional equivalents are members of the IgG immunoglobulin class and subclasses thereof, but may be or may combine with any one of the following immunoglobulin classes: IgM, IgA, IgD, or IgE, and subclasses thereof. Heavy chains of various subclasses, such as the IgG subclasses, are responsible for different effector functions and thus, by choosing the desired heavy chain constant region, hybrid antibodies with desired effector function are produced. Exemplary constant regions are gamma 1 (IgG1), gamma 2 (IgG2), gamma 3 (IgG3), and gamma 4 (IgG4). The light chain constant region can be of the kappa or lambda type.

The immunoglobulins of the present invention can be monovalent, divalent or polyvalent. Monovalent immunoglobulins are dimers (HL) formed of a hybrid heavy chain associated through disulfide bridges with a hybrid light chain. Divalent immunoglobulins are tetramers (H₂L₂) formed of two dimers associated through at least one disulfide bridge.

Combinations

In one embodiment, the composition of the present invention comprises a combination of modulators described herein. For example, in one embodiment the composition comprises two or more inhibitors of miR451a. In one embodiment the composition comprises an inhibitor of miR451a in combination with one or more additional therapeutic agent for the treatment of endometriosis. In certain embodiments, a composition comprising a combination of modulators described herein has an additive effect, wherein the overall effect of the combination is approximately equal to the sum of the effects of each individual modulator. In other embodiments, a composition comprising a combination of modulators described herein has a synergistic effect, wherein the overall effect of the combination is greater than the sum of the effects of each individual modulator.

A composition comprising a combination of modulators comprise individual modulators in any suitable ratio. For example, in one embodiment, the composition comprises a 1:1 ratio of two individual modulators. In another embodiment, the composition comprises a 1:1:1 ratio of three individual modulators. However, the combination is not limited to any particular ratio. Rather any ratio that is shown to be effective is encompassed.

Modified Cell

The present invention includes a composition comprising a cell which comprises or expresses a modulator of the invention. In one embodiment, the cell is genetically modified to express a protein and/or nucleic acid of the invention. In certain embodiments, genetically modified cell is autologous to a subject being treated with the composition of the invention. Alternatively, the cells can be allogeneic, syngeneic, or xenogeneic with respect to the subject. In certain embodiment, the cell is able to secrete or release the modulator into extracellular space in order to deliver the modulator to one or more other cells.

The genetically modified cell may be modified in vivo or ex vivo, using techniques standard in the art. Genetic modification of the cell may be carried out using an expression vector or using a naked isolated nucleic acid construct.

In one embodiment, the cell is obtained and modified ex vivo, using an isolated nucleic acid molecule encoding one or more proteins, miRNA, or other nucleic acid molecule described herein. In one embodiment, the cell is obtained from a subject, genetically modified to express the protein and/or nucleic acid, and is re-administered to the subject. In certain embodiments, the cell is expanded ex vivo or in vitro to produce a population of cells, wherein at least a portion of the population is administered to a subject in need.

In one embodiment, the cell is genetically modified to stably express the modulator. In another embodiment, the cell is genetically modified to transiently express the modulator.

Substrates

The present invention provides a scaffold or substrate composition comprising a modulator of the invention, an isolated nucleic acid of the invention, a cell expressing the modulator of the invention, or a combination thereof. For example, in one embodiment, an inhibitor of the invention, an isolated antisense nucleic acid of the invention, a cell expressing the inhibitor of the invention, or a combination thereof is incorporated within a scaffold. In another embodiment, an inhibitor of the invention, an isolated antisense nucleic acid of the invention, a cell expressing the inhibitor of the invention, or a combination thereof is applied to the surface of a scaffold. The scaffold of the invention may be of any type known in the art. Non-limiting examples of such a scaffold includes a, hydrogel, electrospun scaffold, foam, mesh, sheet, patch, and sponge.

Pharmaceutical Compositions

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, intratumoral, or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents, including, for example, chemotherapeutics, immunosuppressants, corticosteroids, analgesics, and the like.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intrasternal injection, intratumoral, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3 butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, for example, from about 1 to about 6 nanometers. Such compositions are conveniently in the form of dry powders for administration using a device comprising a dry powder reservoir to which a stream of propellant may be directed to disperse the powder or using a self-propelling solvent/powder dispensing container such as a device comprising the active ingredient dissolved or suspended in a low-boiling propellant in a sealed container. For example, such powders comprise particles wherein at least 98% of the particles by weight have a diameter greater than 0.5 nanometers and at least 95% of the particles by number have a diameter less than 7 nanometers. For example, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (e.g., having a particle size of the same order as particles comprising the active ingredient).

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3 butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

Additionally, the molecules may be delivered using a sustained-release system, such as semipermeable matrices of solid polymers containing the therapeutic agent. Various forms of sustained-release materials have been established and are well known by those skilled in the art. Sustained-release capsules may, depending on their chemical nature, release the molecules for a few weeks up to over 100 days. Depending on the chemical nature and the biological stability of the chimeric molecules, additional strategies for molecule stabilization may be employed.

Nucleic acids may be included in any of the above-described formulations as the free acids or bases or as pharmaceutically acceptable salts. Pharmaceutically acceptable salts are those salts that substantially retain the biologic activity of the free bases and which are prepared by reaction with inorganic acids. Pharmaceutical salts tend to be more soluble in aqueous and other protic solvents than are the corresponding free base forms.

In addition to the formulations described previously, the molecules may also be formulated as a depot preparation. Such long acting formulations may be administered by implantation (for example subcutaneously or intramuscularly) or by intramuscular injection. Thus, for example, the molecules may be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems may be employed. Liposomes and emulsions are well-known examples of delivery vehicles that may be used to deliver nucleic acids of the disclosure.

Gene Therapy Administration

One skilled in the art recognizes that different methods of delivery may be utilized to administer a nucleic acid molecule into a cell. Examples include: (1) methods utilizing physical means, such as electroporation (electricity), a gene gun (physical force) or applying large volumes of a liquid (pressure); and (2) methods wherein said molecule is complexed to another entity, such as a liposome, aggregated protein or transporter molecule.

Furthermore, the actual dose and schedule can vary depending on whether the compositions are administered in combination with other pharmaceutical compositions, or depending on inter-individual differences in pharmacokinetics, drug disposition, and metabolism. Similarly, amounts can vary in in vitro applications depending on the particular cell line utilized (e.g., based on the number of vector receptors present on the cell surface, or the ability of the particular vector employed for gene transfer to replicate in that cell line). Furthermore, the amount of nucleic acid molecule to be added per cell will likely vary with the length and stability of the therapeutic gene or miR, as well as the nature of the sequence, and the nature of the molecule (e.g. whether the therapeutic antisense oligonucleotide is incorporated into an expression vector), and is particularly a parameter which needs to be determined empirically, and can be altered due to factors not inherent to the methods of the present invention (for instance, the cost associated with synthesis). One skilled in the art can easily make any necessary adjustments in accordance with the exigencies of the particular situation.

Cells containing the therapeutic agent may also contain a suicide gene i.e., a gene which encodes a product that can be used to destroy the cell. In many gene therapy situations, it is desirable to be able to express a gene for therapeutic purposes in a host, cell but also to have the capacity to destroy the host cell at will. The therapeutic agent can be linked to a suicide gene, whose expression is not activated in the absence of an activator compound. When death of the cell in which both the agent and the suicide gene have been introduced is desired, the activator compound is administered to the cell thereby activating expression of the suicide gene and killing the cell. Examples of suicide gene/prodrug combinations which may be used are herpes simplex virus-thymidine kinase (HSV-tk) and ganciclovir, acyclovir; oxidoreductase and cycloheximide; cytosine deaminase and 5-fluorocytosine; thymidine kinase thymidilate kinase (Tdk::Tmk) and AZT; and deoxycytidine kinase and cytosine arabinoside.

Treatment Methods

The present invention provides methods of treating or preventing endometriosis or an endometriosis-related disease or disorder. In certain embodiments, the method of the invention comprises administering to a subject an effective amount of a composition that inhibits or decreases the level, activity, or both of miR451a in a cell of the subject. In one embodiment, the method of the invention comprises administering to a subject an effective amount of a composition that decreases the activity of miR451a in a cell of the subject.

In one aspect, the invention provides a method of reducing lesion growth in a subject in need thereof. In one embodiment, the invention provides a method of reducing lesion size in a subject in need thereof. In one embodiment, the lesion is an endometriosis lesion. In one embodiment, the invention provides a method of increasing the expression levels of one or more of YHWAZ, CAB39, MAPK1, β-catenin and IL-6 in a subject in need thereof. In one aspect, the invention provides a method of reducing inflammation. In one embodiments, the method of the invention comprises administering to a subject an effective amount of a composition that inhibits or decreases the expression, activity, or both of miR451a in a cell of the subject. In one embodiment, the method of the invention comprises administering to a subject an effective amount of a composition that decreases the activity of miR451a in a cell of the subject. In one embodiment, the method of the invention comprises administering to a subject an effective amount of a composition comprising an antisense oligonucleotide inhibitor of miR451a.

Endometriosis related diseases and disorders include, but are not limited to, ovarian cysts, cancer (such as ovarian cancer, breast cancer, non-Hodgkin's lymphoma, and uterine cancer), uterine fibroids, miscarriage and ectopic pregnancy.

In one embodiment, the subject has endometriosis or an endometriosis-related disease or disorder. For example, in one embodiment, the method comprises administering to a subject having endometriosis or an endometriosis-related disease or disorder an effective amount of a composition that inhibits or decreases the expression, activity, or both of miR451a in a cell of the subject. In one embodiment, the method of the invention comprises administering to a subject having endometriosis or an endometriosis-related disease or disorder an effective amount of a composition that decreases the activity of miR451a in a cell of the subject. In one embodiment, the method of the invention comprises administering to a subject having endometriosis or an endometriosis-related disease or disorder an effective amount of a composition comprising an antisense oligonucleotide inhibitor of miR451a.

In one embodiment, the subject exhibits one or more symptoms of endometriosis or an endometriosis-related disease or disorder. For example, in one embodiment, the method comprises administering to a subject exhibiting one or more symptoms of endometriosis or an endometriosis-related disease or disorder an effective amount of a composition that inhibits or decreases the expression, activity, or both of miR451a in a cell of the subject. In one embodiment, the method of the invention comprises administering to a subject exhibiting one or more symptoms of endometriosis or an endometriosis-related disease or disorder an effective amount of a composition that decreases the activity of miR451a in a cell of the subject. In one embodiment, the method of the invention comprises administering to a subject exhibiting one or more symptoms of endometriosis or an endometriosis-related disease or disorder an effective amount of a composition comprising an antisense oligonucleotide inhibitor of miR451a.

In one embodiment, the subject is identified as having endometriosis based upon the detection of one or more biomarkers indicative of endometriosis. Exemplary biomarkers indicative of endometriosis, and exemplary methods of diagnosing a subject with endometriosis based upon such biomarkers, are described in PCT Publication No.: WO 2015/148919, PCT Publication No.: WO 2018/044979, and PCT Publication No.: WO 2020/092672, each of which is incorporated by reference in their entireties.

In one embodiment, the method comprises diagnosing a subject with endometriosis or an endometriosis-related disease and disorder; and administering to the subject one or more of the inhibitors described herein. For example, in one embodiment, the method comprises diagnosing a subject with endometriosis or an endometriosis-related disease and disorder; and administering to the subject an effective amount of a composition that inhibits or decreases the expression, activity, or both of miR451a in a cell of the subject. In one embodiment, the method of the invention comprises diagnosing a subject with endometriosis or an endometriosis-related disease and disorder; and administering to the subject an effective amount of a composition that decreases the activity of miR451a in a cell of the subject. In one embodiment, the method of the invention comprises diagnosing a subject with endometriosis or an endometriosis-related disease and disorder; and administering to the subject an effective amount of a composition comprising an antisense oligonucleotide inhibitor of miR451a.

The activity of miR451a can be decreased or inhibited using any method known to the skilled artisan. Examples of methods that decrease miR451a activity, include but are not limited to, decreasing the expression of an endogenous gene encoding miR451a, decreasing the expression of miR451a, and decreasing the function, activity, or stability of miR451a. A miR451a inhibitor may therefore be a compound that decreases expression of a gene encoding miR451a, decreases RNA half-life, stability, or decreases miR451a function, activity or stability. In some aspects, the level of miR451a can be decreased by increasing the level or activity of a protein or nucleic acid that degrades or inhibits miR451a. A miR451a inhibitor may be any type of compound, including but not limited to, a polypeptide, a nucleic acid, an aptamer, an anti-miR, antagomiR, a miR sponge, a silencing RNA (siRNA), a short hairpin RNA (shRNA), a morpholino, a piwi-interacting RNA (piRNA), a repeat associated small interfering RNA (rasiRNAs), and a small molecule, or combinations thereof.

Inhibition of miR451a may be accomplished either directly or indirectly. For example, miR451a may be directly inhibited by compounds or compositions that directly interact with miR451a, such as proteins or antisense oligonucleotides. Levels of miR451a may be directly decreased by administering a antisense oligonucleotide that targets miR451a. Alternatively, miR451a may be decreased or inhibited indirectly by compounds or compositions that modulate regulators which inhibit miR451a expression.

Modulating expression of an endogenous gene includes providing a specific modulator of gene expression. Decreasing expression of mRNA or protein includes decreasing the half-life or stability of mRNA or decreasing expression of mRNA. Methods of decreasing expression or activity of miR451a include, but are not limited to, methods that use an siRNA, a miRNA, an antisense nucleic acid, CRISPR guide RNA, a ribozyme, an expression vector encoding a transdominant negative mutant, a peptide, a small molecule, and combinations thereof.

Administration of a composition described herein in a method of treatment can be achieved in a number of different ways, using methods known in the art. It will be appreciated that a composition of the invention may be administered to a subject either alone, or in conjunction with another therapeutic agent.

In some embodiments of the methods for treating or preventing endometriosis in a subject in need thereof, a second agent is administered to the subject. For example, in one embodiment, a second endometriosis therapeutic is administered to the subject.

In another embodiment, the invention provides a method to treat endometriosis comprising treating the subject prior to, concurrently with, or subsequently to the treatment with a composition of the invention, with a complementary therapy for the endometriosis, such as surgery, endometriosis therapeutics, including, but not limited to, hormonal therapy, or a combination thereof.

Exemplary endometriosis therapeutics include, but are not limited to, GnRH agonists, including Leuprolide, Goserelin, Nafarelin, Cetrorelix, Ganirelix, and Elagolix; progestins, including medroxyprogesterone acetate, and norethindrone acetate; danazol; combined oral contraceptives; Etonogestrel/ethinyl E₂ vaginal ring; levonorgestrel IUD; COX-2 inhibitors, including rofecoxib; PPAR-γ agonists, including Rosiglitazone and Pioglitazone; Aromatase inhibitors including Letrozole and Anastrozole; Selective estrogen receptor modulators including Raloxifene; Statins including simvastatin; immunomodulators, including TNFα inhibitors (e.g. infliximab) or TNF inhibitors (e.g. etancercept); and Valproic acid.

Dosing

In one embodiment, a composition is administered to a subject. The composition may also be a hybrid or fusion to facilitate, for instance, delivery to target cells or efficacy. In one embodiment, a hybrid composition may comprise a tissue-specific targeting sequence. For example, in one embodiment, the composition is targeted to uterine cell.

The therapeutic and prophylactic methods of the invention thus encompass the use of pharmaceutical compositions comprising a modulator described herein, or a combination thereof to practice the methods of the invention. The pharmaceutical compositions useful for practicing the invention may be administered to deliver a dose of from ng/kg/day and 100 mg/kg/day. In one embodiment, the invention envisions administration of a dose which results in a concentration of the compound of the present invention from 1 μM and 10 μM in a mammal.

Typically, dosages which may be administered in a method of the invention to a mammal, for example a human, range in amount from 0.5 μg to about 50 mg per kilogram of body weight of the mammal, while the precise dosage administered will vary depending upon any number of factors, including but not limited to, the type of mammal and type of disease state being treated, the age of the mammal and the route of administration. In one embodiment, the dosage of the compound will vary from about 1 μg to about 10 mg per kilogram of body weight of the mammal. In one embodiment, the dosage will vary from about 3 μg to about 1 mg per kilogram of body weight of the mammal.

The compound may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

In one embodiment, the invention includes a method comprising administering a combination of modulators described herein. In certain embodiments, the method has an additive effect, wherein the overall effect of the administering a combination of modulators is approximately equal to the sum of the effects of administering each individual modulator. In other embodiments, the method has a synergistic effect, wherein the overall effect of administering a combination of modulators is greater than the sum of the effects of administering each individual modulator.

The method comprises administering a combination of modulators in any suitable ratio. For example, in one embodiment, the method comprises administering two individual modulators at a 1:1 ratio. In another embodiment, the method comprises administering three individual modulators at a 1:1:1 ratio. However, the method is not limited to any particular ratio. Rather any ratio that is shown to be effective is encompassed.

One exemplary approach provided by the disclosure involves administration of a recombinant therapeutic, such as a recombinant miRNA molecule, variant, or fragment thereof, either directly to the site of a potential or actual disease-affected tissue or systemically (for example, by any conventional recombinant administration technique). The dosage of the administered miRNA depends on a number of factors, including the size and health of the individual patient. For any particular subject, the specific dosage regimes should be adjusted over time according to the individual need and the professional judgment of the person administering or supervising the administration of the compositions.

A miRNA or miRNA mimic may be administered in dosages between about 1 and 100 mg/kg (e.g., 1, 5, 10, 20, 25, 50, 75, and 100 mg/kg).

Nucleic Acid Based Therapies

The disclosure provides isolated miRNAs and nucleic acid molecules encoding such sequences. A recombinant miRNA or a nucleic acid molecule encoding such a miRNA may be administered to reduce the growth, survival, or proliferation of a tumor or neoplastic cell in a subject in need thereof. In one approach, the miRNA is administered as a naked RNA molecule. In another approach, it is administered in an expression vector suitable for expression in a mammalian cell.

A nucleic acid of the disclosure may be administered in combination with a carrier or lipid to increase cellular uptake. For example, the oligonucleotide may be administered in combination with a cationic lipid. Examples of cationic lipids include, but are not limited to, lipofectin, DOTMA, DOPE, and DOTAP. The publication of WO0071096, which is specifically incorporated by reference, describes different formulations, such as a DOTAP: cholesterol or cholesterol derivative formulation that can effectively be used for gene therapy. Other disclosures also discuss different lipid or liposomal formulations including nanoparticles and methods of administration; these include, but are not limited to, U.S. Patent Publication 20030203865, 20020150626, 20030032615, and 20040048787, which are specifically incorporated by reference to the extent they disclose formulations and other related aspects of administration and delivery of nucleic acids. Methods used for forming particles are also disclosed in U.S. Pat. Nos. 5,844,107, 5,877,302, 6,008,336, 6,077,835, 5,972,901, 6,200,801, and 5,972,900, which are incorporated by reference for those aspects.

The nucleic acids may also be administered in combination with a cationic amine such as poly (L-lysine). Nucleic acids may also be conjugated to a chemical moiety, such as transferrin and cholesteryls. In addition, oligonucleotides may be targeted to certain organelles by linking specific chemical groups to the oligonucleotide.

Polynucleotide therapy featuring a nucleic acid molecule encoding a miRNA is another therapeutic approach for treating or preventing endometriosis in a subject. Expression vectors encoding the miRNAs can be delivered to cells of a subject for the treatment or prevention of endometriosis. The nucleic acid molecules must be delivered to the cells of a subject in a form in which they can be taken up and are advantageously expressed so that therapeutically effective levels can be achieved.

Methods for delivery of the nucleic acid molecules to the cell according to the disclosure include using a delivery system, such as liposomes, polymers, microspheres, gene therapy vectors, and naked DNA vectors.

miRNAs may be encoded by a nucleic acid molecule comprised in a vector. The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., BACs and YACs). One of skill in the art would be well equipped to construct a vector through standard recombinant techniques, which are described in Sambrook et al., 2012 and Ausubel et al., 2003, both incorporated herein by reference. Transducing viral (e.g., retroviral, adenoviral, lentiviral and adeno-associated viral) vectors can be used for somatic cell gene therapy, especially because of their high efficiency of infection and stable integration and expression (see, e.g., Cayouette et al., Human Gene Therapy 8:423-430, 1997; Kido et al, Current Eye Research 15:833-844, 1996; Bloomer et al, Journal of Virology 71:6641-6649, 1997; Naldini et al, Science 272:263-267, 1996; and Miyoshi et al, Proc. Natl. Acad. Sci. U.S.A. 94: 10319, 1997). For example, a nucleotide sequence encoding a miRNA molecule can be cloned into a retroviral vector and expression can be driven from its endogenous promoter, from the retroviral long terminal repeat, or from a promoter specific for a target cell type of interest. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244: 1275-1281, 1989; Eglitis et al, BioTechniques 6:608-614, 1988; Tolstoshev et al, Current Opinion in Biotechnology 1:55-61, 1990; Sharp, The Lancet 337: 1277-1278, 1991; Cometta et al, Nucleic Acid Research and Molecular Biology 36:311-322, 1987; Anderson, Science 226:401-409, 1984; Moen, Blood Cells 17:407-416, 1991; Miller et al, Biotechnology 7:980-990, 1989; Le Gal La Salle et al, Science 259:988-990, 1993; and Johnson, Chest 107:77S-83S, 1995). Retroviral vectors are particularly well developed and have been used in clinical settings (Rosenberg et al, N. Engl. J. Med 323:370, 1990; Anderson et al, U.S. Pat. No. 5,399,346).

Other suitable methods for nucleic acid delivery to effect expression of compositions of the present disclosure are believed to include virtually any method by which a nucleic acid (e.g., DNA, including viral and nonviral vectors) can be introduced into an organelle, a cell, a tissue or an organism, as described herein or as would be known to one of ordinary skill in the art.

The administration of a nucleic acid or peptide inhibitor of the invention to the subject may be accomplished using gene therapy. Gene therapy, which is based on inserting a therapeutic gene into a cell by means of an ex vivo or an in vivo technique. Suitable vectors and methods have been described for genetic therapy in vitro or in vivo, and are known as expert on the matter; see, for example, Giordano, Nature Medicine 2 (1996), 534-539; Schaper, Circ. Res 79 (1996), 911-919; Anderson, Science 256 (1992), 808-813; Isner, Lancet 348 (1996), 370-374; Muhlhauser, Circ. Res 77 (1995), 1077-1086; Wang, Nature Medicine 2 (1996), 714-716; WO94/29469; WO97/00957 or Schaper, Current Opinion in Biotechnology 7 (1996), 635-640 and the references quoted therein. The polynucleotide codifying the polypeptide of the invention can be designed for direct insertion or by insertion through liposomes or viral vectors (for example, adenoviral or retroviral vectors) in the cell. In one embodiment, the cell is a cell of the germinal line, an embryonic cell or egg cell or derived from the same. In one embodiment, the cell is a core cell. Suitable gene distribution systems that can be used according to the invention may include liposomes, distribution systems mediated by receptor, naked DNA and viral vectors such as the herpes virus, the retrovirus, the adenovirus and adeno-associated viruses, among others. The distribution of nucleic acids to a specific site in the body for genetic therapy can also be achieved by using a biolistic distribution system, such as that described by Williams (Proc. Natl. Acad. Sci. USA, 88 (1991), 2726-2729). The standard methods for transfecting cells with recombining DNA are well known by an expert on the subject of molecular biology, see, for example, WO94/29469; see also supra. Genetic therapy can be carried out by directly administering the recombining DNA molecule or the vector of the invention to a patient or transfecting the cells with the polynucleotide or the vector of the invention ex vivo and administering the transfected cells to the patient.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. miRNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct the expression of a nucleic acid. The enhancers used can include, without limitation, those that are characterized as tissue- or cell-specific enhancers. For any particular subject, the specific dosage regimes should be adjusted over time according to the individual need and the professional judgment of the person administering or supervising the administration of the compositions.

### EXPERIMENTAL EXAMPLES

The disclosure is further described in detail by reference to the following experimental examples. These examples are provided for purposes of illustration only, and are not intended to be limiting unless otherwise specified. Thus, the disclosure should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present disclosure and practice the claimed methods. The following working examples therefore, specifically point out the preferred embodiments of the present disclosure, and are not to be construed as limiting in any way the remainder of the disclosure.

### Example 1: miR-451a Inhibition Reduces Established Endometriosis Lesions in Mice

The expression of miR-451a in endometriotic lesions and in eutopic endometrium is distinct. Hawkins et al reported that miR-451a expression was elevated in ovarian endometriomas compared with eutopic endometrium (Hawkins et al., Mol Endocrinol. 2011. 25(5): 821-32). Similarly Joshi N R et al demonstrated that miR-451a expression in endometriotic lesions is significantly higher compared to that of the corresponding eutopic endometrium (Joshi et al, Hum Reprod. 2015. 30(12): 2881-91). Similarly, comparing eutopic endometrium with ectopic lesions from women with endometriosis, Graham et al also found that miR-451a is elevated in endometriotic tissue (Graham et al., Hum Reprod. 2015. 30(3): 642-52). In eutopic endometrium of endometriosis patients, the expression of miR-451a is reduced, and this reduction of eutopic endometrium miR-451a expression is associated with disease development (Joshi et al, Hum Reprod. 2015. 30(12): 2881-91). Nothnick W B et al reported that absence of miR-451 is associated with a reduced ability of endometrial tissue to establish ectopically in a murine experimental model for endometriosis (Nothnick et al., PLoS One. 2014. 9(6): e100336). Marked down-regulation of miR-451a was also demonstrated in the eutopic endometrium of baboons (Joshi et al., Hum Reprod. 2015. 30(12): 2881-91). Thus, there are contrasting findings with respect to the expression and function of miR-451a in the ectopic and eutopic endometrium.

The goal of this project was to evaluate whether alteration of miR-451a could have a role in the treatment of endometriosis (Graham et al., Hum Reprod. 2015. 30(3): 642-52). Here, the therapeutic use of a miR-451a inhibitor in the treatment of endometriosis using a murine model is demonstrated. miR-451a has been demonstrated to regulate expression of genes which are thought to modulate these physiological events conducive to endometriotic implant establishment and/or survival. These include macrophage migration inhibitory factor (MIF) and 14-3-3 protein zeta (YWHAZ) (Graham et al., Hum Reprod. 2015. 30(3): 642-52; Trattnig et al. PLoS One. 2018. 13(11): e0207575). MIF is a cytokine which is secreted by endometriotic cells in vitro and exhibits mitogenic activity, promoting the growth of endothelial cells (Graham et al., Hum Reprod. 2015. 30(3): 642-52). Other genes not previously associated with endometriois were also altered. There are no data available in literature with regard to CAB39 in endometriosis, however in colorectal cancer, its presence is connected to the regulation of cell proliferation and poor overall survival (Ruhl et al., BMC Cancer. 2018. 18(1): 517). In other diseases, miR-451 directly inhibited expression of β-catenin and subsequently markedly inhibited downstream indirect target genes cyclin D1, and CAB39, IL-6 (Trattnig et al. PLoS One. 2018. 13(11): e0207575; Wang et al., Cell Death Dis. 2017. 8(10): e3071; Ruhl et al., BMC Cancer. 2018. 18(1): 517; Sun et al., Cell Tissue Res. 2018. 374(3): 487-495). All of these genes have been shown to be associated with cellular proliferation and survival, with MIF and YWHAZ previously being examined in endometriotic tissue and cells. This study demonstrates that all were increased by treatment with the miR-451a inhibitor.

Suppressing the elevated miR-451a in the serum by treatment with an inhibitor suppressed lesion growth while paradoxically increasing proliferative markers in the lesions. Previously widespread systemic effects of endometriosis associated microRNAs have been demonstrated (Nematian et al., J Clin Endocrinol Metab. 2018. 103(1): 64-74). Without being bound by theory, it was postulated that the elevated serum miR-451a in endometriosis functions not only by controlling growth of endometrium and lesions; it also has widespread systemic effects that likely enable lesion development. The extent of these alterations are not fully known but may include modulation of immune function, angiogenesis and eutopic endometrial proliferation.

Previously it was reported that local treatment of endometriosis with Let-7b is a promising therapy for endometriosis that simultaneously affects multiple pathways driving endometriosis without systemic hormonal side effects (Sahin et al., J Cell Mol Med. 2018). The studies presented here found that miR-451a inhibition treatment decrease the size of endometriosis lesions in mice. It is also demonstrated that the expression of target genes of miR-451a are increased significantly. Administration here was by the intravenous rather than local route, making it more amenable for human therapy. MiR-451a inhibitors may be an easily administered effective therapy for endometriosis that does not lead to the hormonal side effects associated with current therapies.

The materials and methods employed in these experiments are now described.

Animals

Six- to eight-week-old C57BL/6J wild-type female mice were purchased from Jackson Laboratories (Bar Harbor, Me., USA). Mice were maintained in the animal facility of Yale School of Medicine. Animals were housed per cage in a 12-hour light, 12-hour dark cycle (7 AM-7 PM) with ad libitum access to food and water. All animals were treated under an approved protocol by Yale University Institutional Animal Care and Use Committee. Mice were acclimated for at least 1 week, and vaginal cytology analysis was performed to determine estrous cycle stage of individual animals prior to surgery. All recipient and donor animals were in diestrous stage at the time of endometriosis induction.

Experimental Murine Endometriosis

Endometriosis was induced in twelve mice using a modified version of the syngeneic endometriosis protocol that has been used previously in our laboratory (Lee et al., Biol Reprod. 2009. 80(1): 79-85). In accordance with this model, identically sized uterine tissue fragments were sutured onto the peritoneal surface. Six donor mice in diestrous stage were euthanized using a CO2 chamber, both uterine horns from each mouse were removed and opened longitudinally with microscissors under a stereo-microscope (M651; Leica Microsystems GmbH, Wetzlar, Germany) and divided into equal fragments measuring 2 mm by dermal biopsy punch. These fragments were preserved on ice in DMEM/F12 Ham 1:1 media (Gibco; Grand Island, N.Y., USA) until transplantation. For implantation, recipient mice were anaesthetized by inhalation of isoflurane (Isothesia; Henry Schein, OH, USA) and laparotomy was performed by midline incision. Four identically sized uterine fragments were sutured to the right and left peritoneal surface using 5-0 polyglactin sutures (Vicryl; Ethicon, Somerville, N.J., USA) with the perimetrium adjacent to the peritoneum, and at identical positions of the abdominal wall. Subsequently, the peritoneum and skin were closed using the same suture material. Animals were treated with 1 mg/kg/day SQ of Meloxicam as an analgesic for 72 hours post-operatively.

microRNA 451a Inhibitor Treatment

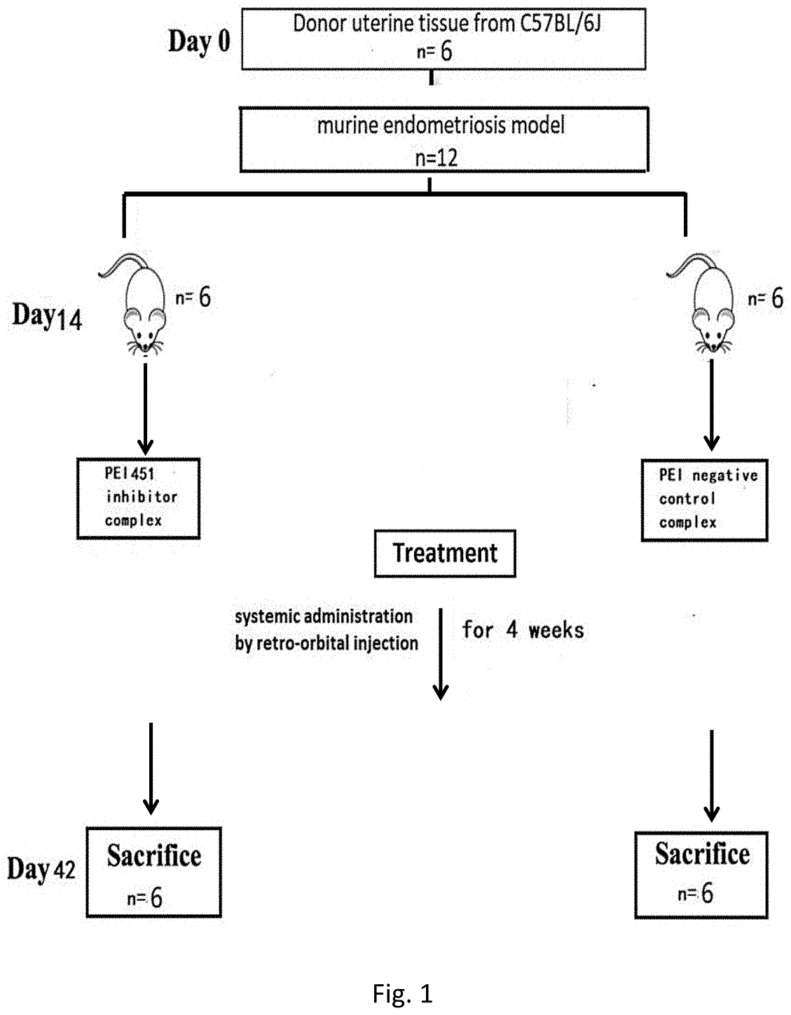

Twelve animals with experimentally induced endometriosis were randomly divided into two groups of six mice in each. Four weeks after the induction of endometriosis, miRNA 451a treatment was initiated with either miR-451a inhibitor (AAACCGUUACCAUUACUGAGUU; SEQ ID NO:1) or miRNA cel-miR-67-3p (UCACAACCUCCUAGAAAGAGUAGA, mirBase accession number: MIMAT0000039; SEQ ID NO:2) as a control. These miRNAs were purchased from W. M. Keck Oligonucleotide Synthesis Facility (Yale University, New Haven, Conn., USA). miRNAs were injected systemically using in vivo-jetPEI carrier (Polyplus-transfection, Illkirch, France). The oligonucleotide+in vivo-jetPEI mixture was prepared according to the manufacturer's guidelines for retro-orbital oligonucleotide injection. Accordingly, 200 ul 5% glucose mixture including 40 μg nucleic acid and 6.4 μL carrier reagent (N/P=8) was prepared for each injection, and mice were treated by retro-orbital injection every 3 days for 4 weeks as shown in FIG. 1.

Macroscopic and Microscopic Evaluation of Lesions and Tissue Collection

After 4 weeks of treatment, animals were euthanized within a CO2 chamber and endometriotic lesions were removed from the peritoneum. All lesions were individually measured, and lesion's volumes were calculated with using (smallest diameter2×largest diameter)*i/6 formula (mm³) (Laschke et al., Am J Pathol. 2010. 176(2): 585-93). Two lesions from each animal were kept in RNA stabilization solution (RNA later; Qiagen, Hilden, Germany) for mRNA isolation to determine the gene expression by qRT-PCR analysis. After H&E staining, all lesions were evaluated under light microscope to confirm endometriosis.

RNA Isolation

Each specimen was thawed on ice, minced and homogenized in 1.0 mL of TRIzol reagent (Invitrogen, Carlsbad, Calif., USA). RNA chloroform extraction was followed by precipitation in isopropyl alcohol and then dissolved in 30 μL of RNase-free water. The total RNA was purified using the RNeasy cleanup kit (Qiagen, Valencia, Calif., USA), according to the manufacturers protocol. The yield of RNA was determined with the use of a Nanodrop ND-2000 spectrophotometer (Nanodrop Technologies). Only RNA samples with appropriate size distribution, quantity, and an A260:A280 ratio of 1.8-2.1 were used for further analysis.

Quantitative Real-Time Polymerase Chain Reaction (qRT-PCR)

Purified RNA was immediately used for cDNA synthesis or stored at −80° C. until use later. For cDNA synthesis, purified RNA (1000 ng) was reverse-transcribed using iScript cDNA synthesis kit (Bio-Rad Laboratories, Hercules, Calif., USA). Real-time quantitative PCR (real-time qPCR) was performed using SYBR Green (Bio-Rad) and optimized in the MyiQ single-color real-time PCR detection system (Bio-Rad). Primer sequences used for gene expression are listed in Table 1. The specificity of the amplified transcript and absence of primer dimers were confirmed by a melting curve analysis. Gene expression was normalized to that of (3-actin as an internal control. Relative mRNA expression was calculated using the comparative cycle threshold (Ct) method (2^{-ΔΔCT}). All experiments were carried out three times and each in duplicate.

TABLE 1
Primer Sequences Used for qRT-PCR.

| Gene | Forward Sequence | SEQ ID NO: | Reverse Sequence | SEQ ID NO: |
|---|---|---|---|---|
| YWHAZ | CAGTAGATGGAGAAAG | 3 | GGGACAATTAGGGA | 4 |
| | ATTTGC | | AGTAAGT | |
| MIF | TGCCCAGAACCGCAAC | 5 | TCGCTACCGGTGGAT | 6 |
| | TACAGTAA | | AAACACAGA | |
| CAB39 | TGCAGGTCTGTGCAGT | 7 | AACAATCCCTGTATG | 8 |
| | ATGG | | CGCCA | |
| MAPK1 | AATTGGTCAGGACAAG | 9 | GAGTGGGTAAGCTG | 10 |
| | GGCTC | | AGACGG | |
| CyclinD1 | AAGTGCGTGCAGAAGG | 11 | GGATAGAGTTGTCA | 12 |
| | AGATTGT | | GTGTAGATGC | |
| TLR-4 | TTCAGAACTTCAGTGGC | 13 | CCATGCCTTGTCTTC | 14 |
| | TGGATT | | AATTGTTT | |
| IL-6 | TAGTCCTTCCTACCCCA | 15 | TTGGTCCTTAGCCAC | 16 |
| | ATTTCC | | TCCTTC | |
| B-catenin | ACTTGCCACACGTGCA | 17 | ATGGTGCGTACAAT | 18 |
| | ATTC | | GGCAGA | |
| TNF-a | AAGCCTGTAGCCCACG | 19 | GGCACCACTAGTTG | 20 |
| | TCGTA | | GTTGTCTTTG | |

Statistical Analysis

GraphPad Prism 7.0 a software (GraphPad Software, La Jolla, Calif., USA) was used for all statistical analyses. All in vitro experiments were performed in triplicate, and the mean for each individual animal was used for statistical analysis. The quantitative data were tested for normality using the Shapiro-Wilk test. Independent-sample t test was used for evaluating of normally distributed variables. Non-normally distributed continuous variables were compared using Mann-Whitney U test. P<0.05 was considered as statistically significant.

The results of the experiments are now described.

miR-451a Inhibitor Treatment and Evaluation of the Lesion

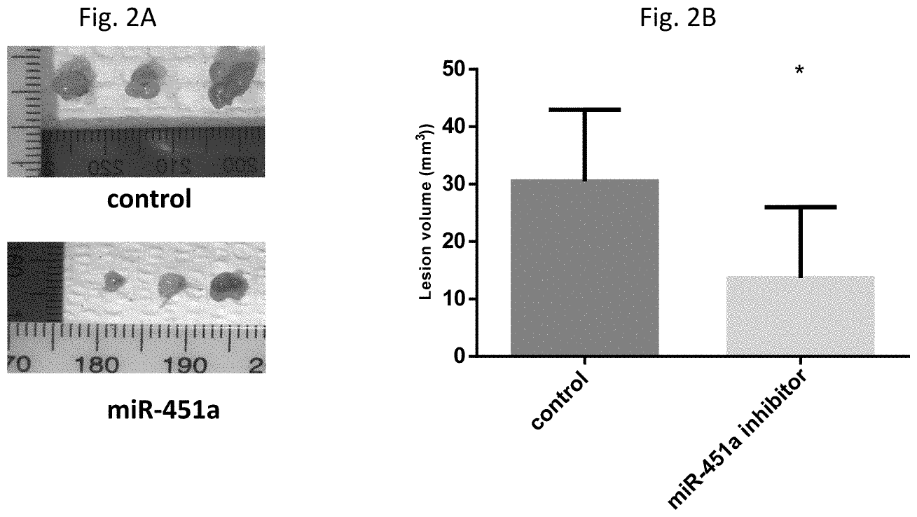

No adverse reactions including weight loss or behavioral alterations were noted in any of the miR-451a inhibitor treated mice. At the end of the miR-451a inhibitor treatment period (4 weeks) mice were euthanized and endometriotic lesions were collected. All the lesions were confirmed using H&E staining. There was no significant difference in the number of lesions between the miR-451a inhibitor treatment and control groups. All of the lesions were cystic. Lesion size and volume were compared between the miR-451a inhibitor treated and control groups. Gross lesion size was lower in the miR-451a inhibitor treated group (13.65±3.71 mm³) than control group (30.26±3.93 mm³; P=0.004) (FIG. 2A and FIG. 2B).

Differential Expression of Genes that are Involved Endometriosis

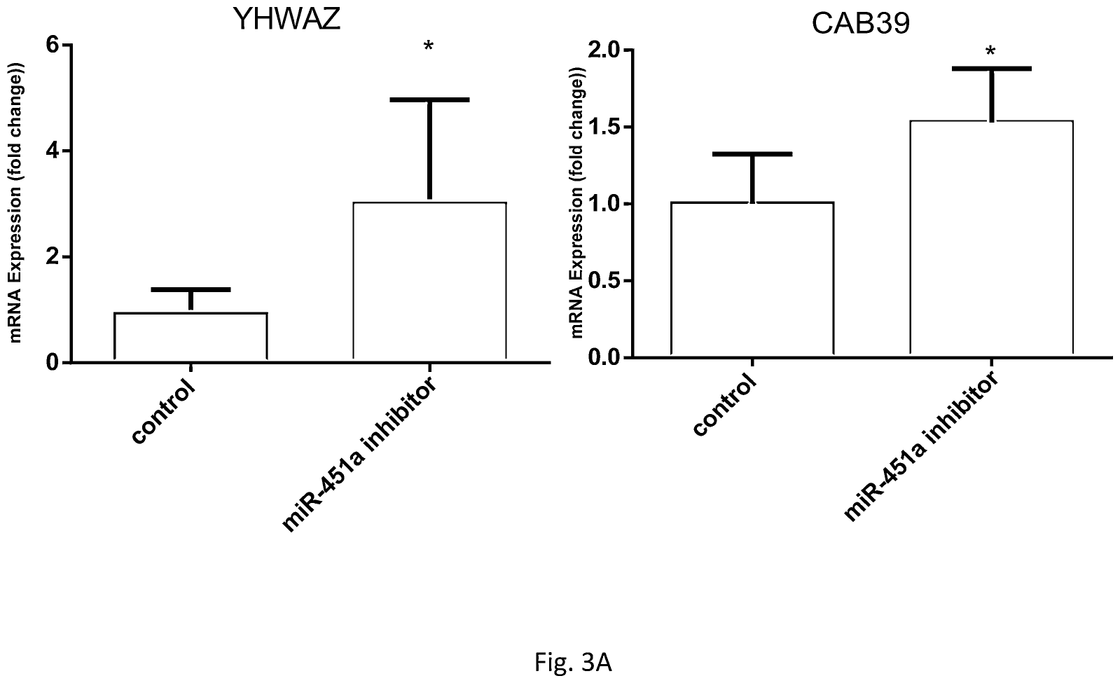

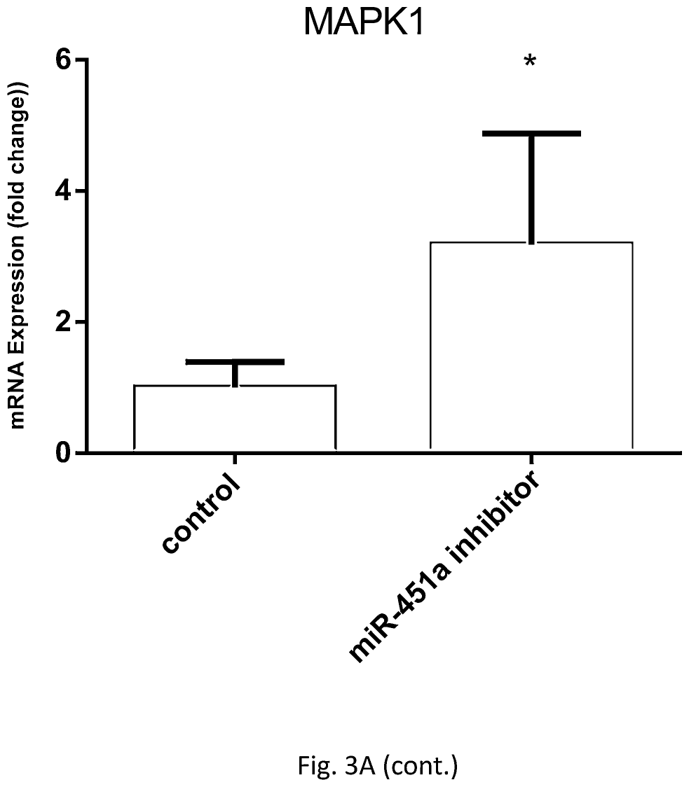

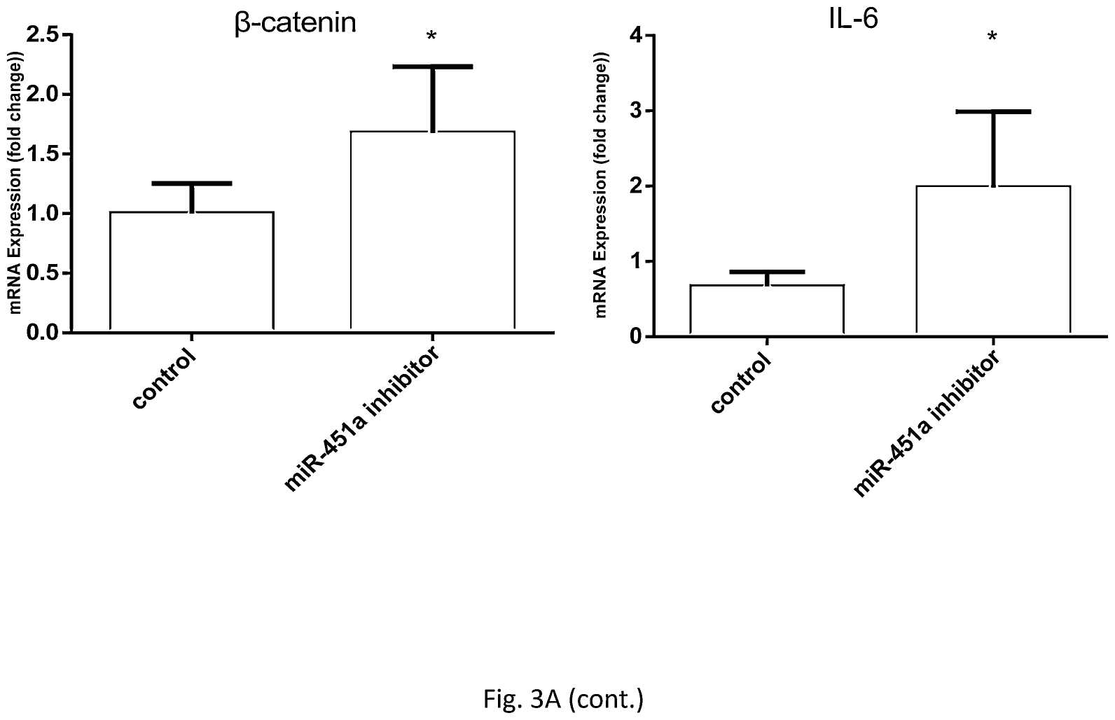

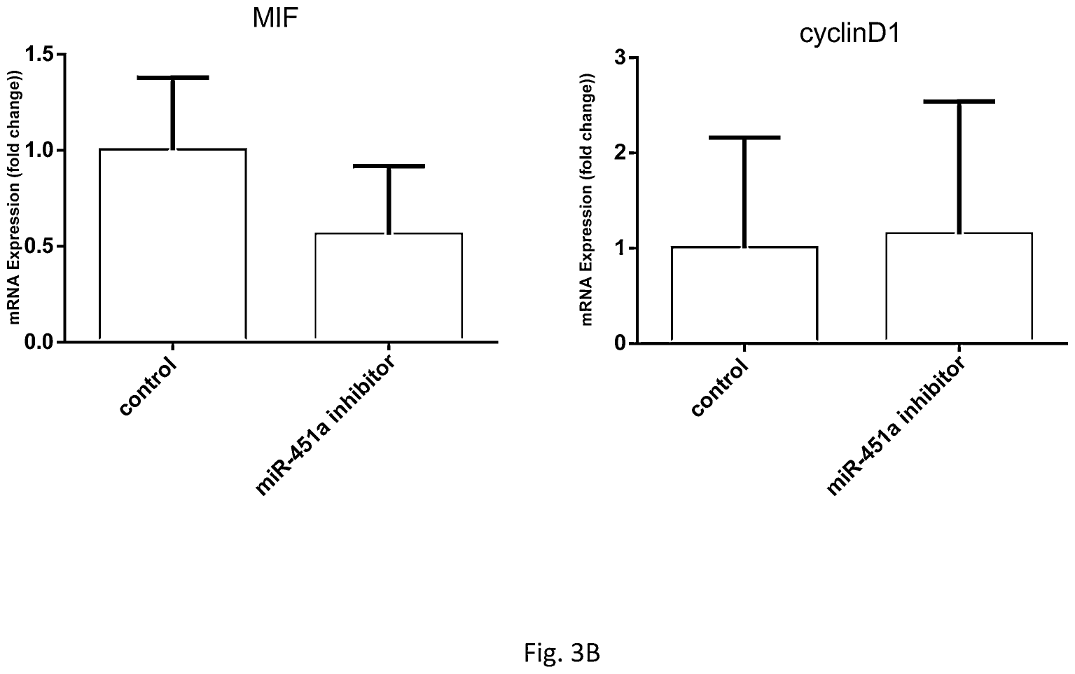

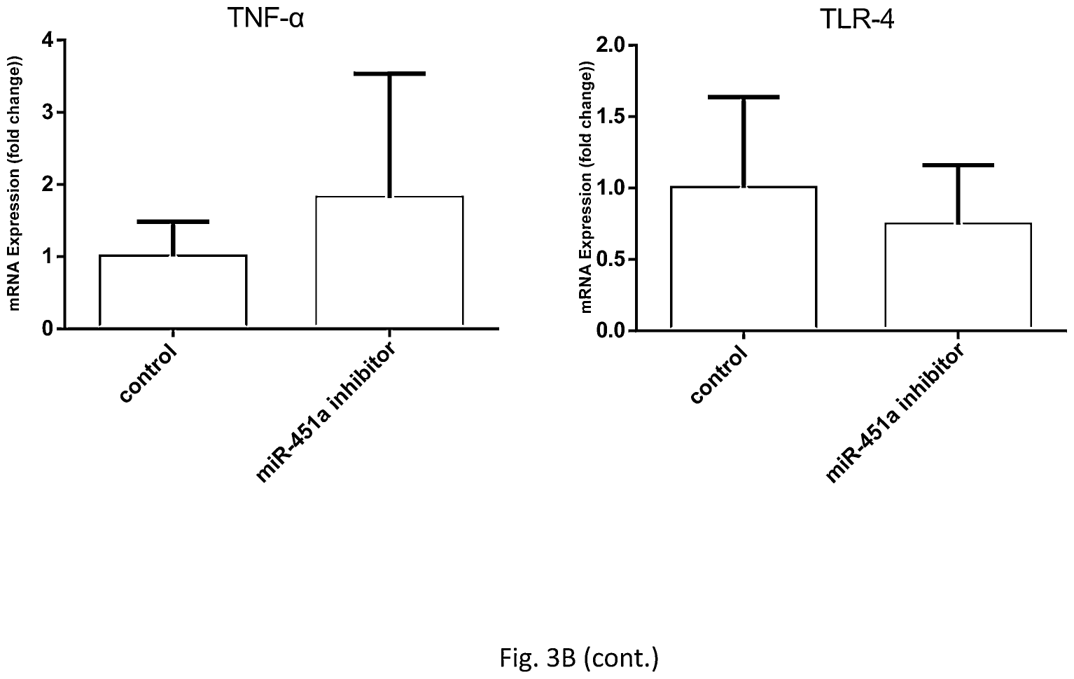

The effect of miR-451a inhibitor treatment on expression of genes that are involved in endometriosis was determined by qRT-PCR in the lesions and compared with expression in the control group. Increased expression of several genes known to mediate endometriosis growth or endometriosis-associated inflammation (Kim et al., Hum Reprod. 2015. 30(5): 1069-78) was observed. Expression of YWHAZ, CAB39, MAPK1 and β-catenin, and IL-6 was increased in the miR-451a inhibitor treatment group compared to the control group. The quantitative increase in gene expression was 3.1-fold (P=0.042) for YWHAZ, 1.5-fold (P=0.030) for CAB39, 3.2-fold (P=0.025) for MAPK1, 1.7-fold (P=0.044) for β-catenin, and 2.0-fold (P=0.021) for IL-6 in inhibitor-treated group compared to the control group as shown in FIG. 3A. Expression levels of the MIF, cyclin-D1, TNF-α and TLR-4 were unchanged between the two groups (P>0.05) as shown in FIG. 3B.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the disclosure described herein may be employed in practicing the invention. It is intended that the following claims define the scope of the invention and that methods and structures within the scope of these claims and their equivalents be covered thereby.

What is claimed is:

1. A method of treating or preventing endometriosis in a subject in need thereof, comprising administering to the subject an effective amount of an inhibitor of microRNA 451a (miR451a).

2. The method of claim 1, wherein the inhibitor is at least one selected form the group consisting of a polypeptide, a nucleic acid, an aptamer, an anti-miR, antagomiR, a miR sponge, a silencing RNA (siRNA), a short hairpin RNA (shRNA), a morpholino, a piwi-interacting RNA (piRNA), a repeat associated small interfering RNA (rasiRNAs), and a small molecule.

3. The method of claim 2, wherein the inhibitor is an antisense nucleic acid molecule to miR451a.

4. The method of claim 3, wherein the inhibitor comprises the sequence AAACCGUUACCAUUACUGAGUU (SEQ ID NO:1).

5. A composition for treating endometriosis comprising an inhibitor of microRNA 451a (miR451a).

6. The composition of claim 5, wherein the inhibitor is at least one selected form the group consisting of a polypeptide, a nucleic acid, an aptamer, an anti-miR, antagomiR, a miR sponge, a silencing RNA (siRNA), a short hairpin RNA (shRNA), a morpholino, a piwi-interacting RNA (piRNA), a repeat associated small interfering RNA (rasiRNAs), and a small molecule.

7. The composition of claim 6, wherein the inhibitor is an antisense nucleic acid molecule to miR451a.

8. The composition of claim 7, wherein the inhibitor comprises the sequence AAACCGUUACCAUUACUGAGUU (SEQ ID NO:1).


SEQUENCE LISTING (20 sequences)

SEQ ID NO: 1 - RNA, 22 nt, Artificial Sequence
  Chemically Synthesized
aaaccguuaccauuacugaguu

SEQ ID NO: 2 - RNA, 24 nt, Artificial Sequence
  Chemically Synthesized
ucacaaccuccuagaaagaguaga

SEQ ID NO: 3 - DNA, 22 bp, Artificial Sequence
  Chemically Synthesized
cagtagatggagaaagatttgc

SEQ ID NO: 4 - DNA, 21 bp, Artificial Sequence
  Chemically Synthesized
gggacaattagggaagtaagt

SEQ ID NO: 5 - DNA, 24 bp, Artificial Sequence
  Chemically Synthesized
tgcccagaaccgcaactacagtaa

SEQ ID NO: 6 - DNA, 24 bp, Artificial Sequence
  Chemically Synthesized
tcgctaccggtggataaacacaga

SEQ ID NO: 7 - DNA, 20 bp, Artificial Sequence
  Chemically Synthesized
tgcaggtctgtgcagtatgg

SEQ ID NO: 8 - DNA, 20 bp, Artificial Sequence
  Chemically Synthesized
aacaatccctgtatgcgcca

SEQ ID NO: 9 - DNA, 21 bp, Artificial Sequence
  Chemically Synthesized
aattggtcaggacaagggctc

SEQ ID NO: 10 - DNA, 20 bp, Artificial Sequence
  Chemically Synthesized
gagtgggtaagctgagacgg

SEQ ID NO: 11 - DNA, 23 bp, Artificial Sequence
  Chemically Synthesized
aagtgcgtgcagaaggagattgt

SEQ ID NO: 12 - DNA, 24 bp, Artificial Sequence
  Chemically Synthesized
ggatagagttgtcagtgtagatgc

SEQ ID NO: 13 - DNA, 23 bp, Artificial Sequence
  Chemically Synthesized
ttcagaacttcagtggctggatt

SEQ ID NO: 14 - DNA, 23 bp, Artificial Sequence
  Chemically Synthesized
ccatgccttgtcttcaattgttt

SEQ ID NO: 15 - DNA, 23 bp, Artificial Sequence
  Chemically Synthesized
tagtccttcctaccccaatttcc

SEQ ID NO: 16 - DNA, 21 bp, Artificial Sequence
  Chemically Synthesized
ttggtccttagccactccttc

SEQ ID NO: 17 - DNA, 20 bp, Artificial Sequence
  Chemically Synthesized
acttgccacacgtgcaattc

SEQ ID NO: 18 - DNA, 20 bp, Artificial Sequence
  Chemically Synthesized
atggtgcgtacaatggcaga

SEQ ID NO: 19 - DNA, 21 bp, Artificial Sequence
  Chemically Synthesized
aagcctgtagcccacgtcgta

SEQ ID NO: 20 - DNA, 24 bp, Artificial Sequence
  Chemically Synthesized
ggcaccactagttggttgtctttg